(19) United States
(12) Patent Application Publication
(10) Pub. No.: US 2026/0232189 A1
(43) Pub. Date: Aug. 13, 2026

(54) IN VIVO BIOMECHANICAL STRAIN RESPONSE OF THE LAMINA CRIBROSA TO INTRAOCULAR PRESSURE CHANGE AS A RESULT OF GLAUCOMA MEDICATION CHANGE

(71) Applicant: THE JOHNS HOPKINS UNIVERSITY, Baltimore, MD (US)

(72) Inventors: Thao D. NGUYEN, Baltimore, MD (US); Harry QUIGLEY, Baltimore, MD (US); Cameron CZERPAK, Baltimore, MD (US); Vanessa HANNAY, Baltimore, MD (US); Dan E. MIDGETT, Baltimore, MD (US)

(21) Appl. No.: 19/155,707

(22) PCT Filed: Jan. 30, 2024
(86) PCT No.: PCT/US2024/013527

§ 371 Date: Aug. 12, 2025

Related U.S. Application Data
(60) Provisional application No. 63/485,590, filed on Feb. 17, 2023.

(51) Int. Cl. A61B 3/16; A61B 3/00; A61B 3/10; A61B 5/00

(52) U.S. Cl. CPC A61B 3/165; A61B 3/0025; A61B 3/102; A61B 5/4848

(57) ABSTRACT

A method and a system that performs the method to characterize in vivo biomechanical response of a lamina cribrosa (LC) to intraocular pressure (IOP) change as a result of a change in glaucoma medication is disclosed. The method includes acquiring a plurality of images of an eye of a patient; modeling the plurality of images; determining a biomechanical response of the LC to IOP change based on the modeling; and determining a course of treatment and ongoing monitoring using the biomechanical response of the LC to IOP change as an aid in detection and management of glaucoma.

### CROSS REFERENCE TO RELATED APPLICATION

This application is the national stage entry of International Patent Application No. PCT/US2024/013527, filed on Jan. 30, 2024, and published as WO 2024/173032 A1 on Aug. 22, 2024, which claims the benefit of U.S. provisional patent application 63/485,590 filed on Feb. 17, 2023, which are hereby incorporated by reference in their entireties.

### GOVERNMENT SUPPORT

This invention was made with government support under grant no. 1727104 awarded by the National Science Foundation and grant no. EY021500 awarded by the National Institutes of Health. The government has certain rights in the invention.

### FIELD OF THE DISCLOSURE

The present disclosure relates to in vivo biomechanical strain response of the lamina cribrosa to intraocular pressure change as a result of glaucoma medication change.

### BACKGROUND

Glaucoma is the leading cause of irreversible blindness. It is estimated that Primary Open Angle Glaucoma (POAG) affects 58 million people worldwide with the number of cases expected to nearly double by 2040. Glaucoma is significantly associated with high levels of intraocular pressure (IOP) which damage the retinal ganglion cell (RGC) axons of the optic nerve over time, resulting in irreversible, progressive vision loss if left untreated.

Glaucoma may be categorized broadly into open-angle (POAG) and angle-closure glaucoma. POAG is the most common form of glaucoma, affecting over 80% of glaucoma patients in the US. The present study focuses on eyes with POAG since it is the most prevalent form of glaucoma, and its underlying mechanism is less well understood, but the method described could apply to all forms of glaucoma. The severity of POAG can be gauged by several clinical measures including average retinal nerve fiber layer (RNFL) thickness, mean deviation (MD), and visual field index (VFI). A thinner RNFL, more negative mean deviation, and lower visual field index are associated with increased damage and disease progression.

Currently, there is no cure for POAG. Vision loss due to glaucoma damage cannot be completely stopped or reversed, however, progression can be slowed. This is best accomplished through IOP-lowering treatments. Treatment options to reduce IOP in glaucoma patients range in invasiveness from medication (topical eye drops) to laser procedures to surgical intervention. If detected and managed early, severe vision loss from glaucoma may be prevented. More often than not, POAG progresses slowly and is asymptomatic prior to vision loss and goes undetected while damage to the optic nerve accumulates.

### SUMMARY

According to examples of the present disclosure, a method to characterize in vivo biomechanical response of a lamina cribrosa (LC) to intraocular pressure (IOP) change as a result of a change in glaucoma medication is disclosed. The method comprises acquiring a plurality of images of an eye of a patient; modeling the plurality of images; determining a biomechanical response of the LC to IOP change based on the modeling; and suggesting the intensity of a course of treatment and ongoing monitoring using the biomechanical response of the LC to IOP change as an aid in detection and management of glaucoma.

According to examples of the present disclosure, a system is described to characterize in vivo biomechanical response of a lamina cribrosa (LC) to intraocular pressure (IOP) change as a result of a change in glaucoma medication. The system comprises an imaging system to acquire a plurality of images of an eye of a patient; a computer system that is configured to perform a method comprising modeling the plurality of images; determining a biomechanical response of the LC to IOP change based on the modeling; and suggesting the intensity of a course of treatment and ongoing monitoring using the biomechanical response of the LC to IOP change as an aid in detection and management of glaucoma.

Various additional features can be included in the method and the system including one or more of the following features. The biomechanical response is short-term biomechanical strain response. The short-term biomechanical strain response is about one-two week. The treatment comprises glaucoma medication, such as IOP-lowering eye drops. The plurality of images comprises optical coherence tomography (OCT) scans. The method can further comprise stacking consecutive OCT scans to construct a 3D image volume. The OCT scans comprise a brightness scan (B-scan), wherein the B-scan comprises data from scanning a sample laterally at multiple points that results in a grayscale cross-sectional image where a grayscale value at each point represents a brightness amplitude of light reflected from a portion of the eye. The OCT scans comprise acquired B-scans spaced at equal intervals circumferentially around an optic nerve head that results in 2D cross-sectional images in which a height of a scan represented an axial direction and a length of the scan represented a radial direction. The OCT scans comprise spectral domain OCT (SDOCT). The modeling comprises performing a digital volume correlation (DVC) operation. The modeling comprises modeling a relationship of LC strains and strain compliance to measures of glaucoma damage, wherein the measures of glaucoma damage comprise RNFL thickness, mean deviation (MD), and visual field index (VFI).

### BRIEF DESCRIPTION OF THE FIGURES

FIG. 1 shows an IOL Master, a standard instrument widely used in ophthalmic clinics, was used to measure the axial length and curvature of each eye.

FIG. 2 shows a Heidelberg Spectralis was used at each imaging session to acquire radial OCT image volumes consisting of 24 slices per volume.

FIG. 3A, FIG. 3B, FIG. 3C show radial OCT scans of the ONH were acquired using the Heidelberg OCT. Each OCT image volume consisted of 24 evenly spaced slices, where FIG. 3A shows an original scan and orientation map, FIG. 3B shows an original OCT image after cropping in Matlab, and FIG. 3C shows an image processed with contrast limited adaptive histogram equalization (CLAHE) and a Gamma filter to enhance contrast of the speckle pattern to optimize DVC correlation.

FIG. 4A and FIG. 4B shows an optic nerve head in each of the 24 slices in every OCT volume was segmented by hand in ImageJ to define the boundaries of the anterior lamina cribrosa (ALC), retina, choroid, and sclera. The posterior border of the ALC was segmented automatically in MATLAB by drawing a border 250 μm below the anterior border of the ALC. The pixel coordinates were imported into MATLAB for DVC post-processing analysis.

FIG. 5A and FIG. 5B show plots of IOP decrease for each test group where after an average of one week of starting glaucoma medication (hypotensive eye drops), IOP decreased in some but not all medication change eyes. The entire medication change group was subdivided into two groups based on IOP decrease magnitude. Group 1 contained eyes with an IOP change of at 4 mmHg while Group 2 contained eyes whose IOP did not change after the treatment period (defined as an IOP change of 0-1 mmHg). No eyes underwent an IOP change between 1 and 4 mmHg. FIG. 5A shows a plot of IOP decrease magnitude and FIG. 5B shows a plot of IOP percent decrease.

FIG. 6 shows plots of anterior lamina depth (ALD) change (μm) after IOP change induced via glaucoma medication change was calculated using DVC. A positive ALD change represents posterior displacement of the ALC border while a negative ALD change represents anterior displacement. An asterisk indicates the group mean was significantly different from zero (p-value less than or equal to 0.05). The median ALD change was positive for all groups and was significant for the entire medication change group and Group 1, suggesting on average the ALC border moved posteriorly after IOP change over the course of one week.

FIG. 7A, FIG. 7B, and FIG. 7C show plots of ALC strains as a result of medication-change induced IOP change for each group. An asterisk (*) indicates p-value less than or equal to 0.05. FIG. 7A shows a plot for the entire medication change group, FIG. 7B shows a plot for Group 1, and FIG. 7C shows a plot for Group 2. IOP decrease produced by hypotensive eye drops resulted in significant tensile E_{zz} and compressive Eᵣᵣ for Group 1. For Group 2, E_{zz} was relatively small compared to E_{zz} of Group 1 and Eᵣᵣ was not significant.

FIG. 8A, FIG. 8B, FIG. 8C, FIG. 8D, FIG. 8E, FIG. 8F, FIG. 8G, and FIG. 8H show plots of DVC strain maps of the ONH after IOP change from medication change were generated in MATLAB. These sample strain maps were taken from a Group 1 eye with an IOP decrease of −7 mmHg and ALC % correlation of 64%. FIG. 8A shows Eₘₐₓ strain map for inferior-superior slice FIG. 8B shows Eₘₐₓ strain map for nasal-temporal slice FIG. 8C shows Γₘₐₓ strain map for inferior-superior slice FIG. 8D shows Γₘₐₓ strain map for nasal-temporal slice FIG. 8E shows E_{zz} strain map for inferior-superior slice FIG. 8F shows E_{zz} strain map for nasal-temporal slice FIG. 8G shows Eᵣᵣ strain map for inferior-superior slice FIG. 8H shows Eᵣᵣ strain map for nasal-temporal slice.

FIG. 9A, FIG. 9B, FIG. 9C, FIG. 9D, FIG. 9E, FIG. 9F, FIG. 9G, and FIG. 9H show plots of strain magnitudes increased with greater IOP decrease and greater percent IOP decrease in the medication change eyes of Group 1. Greater strain magnitudes were more associated with percent IOP decrease than with IOP decrease magnitude. FIG. 9A shows maximum principal strain, Eₘₐₓ, increased with greater IOP decrease. FIG. 9B shows maximum principal strain, Eₘₐₓ, increased with greater percent IOP decrease. FIG. 9C shows maximum shear strain, Γₘₐₓ, increased with greater IOP decrease. FIG. 9D shows maximum shear strain, Γₘₐₓ, increased with greater percent IOP decrease. FIG. 9E shows compressive Eᵣᵣ increased with greater IOP decrease. FIG. 9F shows compressive Eᵣᵣ increased with greater percent IOP decrease. FIG. 9G shows ALD did not change with IOP decrease magnitude. FIG. 9H shows ALD did not change with percent IOP decrease.

FIG. 10A, FIG. 10B, FIG. 10C, FIG. 10D, FIG. 10E, FIG. 10F, FIG. 10G, FIG. 10H, and FIG. 10I show plots of linear regression analysis was used to test whether strain compliance response to IOP change was associated with glaucoma damage. In Group 1, more compliant strain response for Eₘₐₓ, Γₘₐₓ, and E_{zz} was associated with a thicker average RNFL, however, a more compliant strain response for Eₘₐₓ, Γₘₐₓ, and VFI was associated with lower MD and lower VFI.

FIG. 11A, FIG. 11B, and FIG. 11C show plots of linear regression tested whether strain compliance response was correlated with age in Group 1, where FIG. 11A shows strain compliance response for E_{zz}, FIG. 11B shows strain compliance response for Eᵣᵣ, FIG. 11C shows ALD change compliance response. Regression analysis suggested there was a greater compressive Eᵣᵣ strain compliance response with younger age, however this trend appears to be anchored by an age outlier with an age of 30 years old. The E_{zz} strain and ALD change compliance responses were not correlated with age.

FIG. 12 shows a flowchart for a method according to examples of the present disclosure.

FIG. 13 shows an example computer system according to examples of the present disclosure.

### DETAILED DESCRIPTION

The exact mechanism by which IOP level affects vision loss has not yet been elucidated. However, there is a well-documented correlation between the level of IOP and RGC axon loss which has been demonstrated in experimental glaucoma models and randomized human clinical trials. As previously stated, while IOP level remains a significant risk factor for glaucoma, not all individuals with ocular hypertension (defined as IOP greater than 21 mmHg) develop glaucoma. In fact, recent findings suggest the majority of individuals with ocular hypertension do not develop glaucoma. Furthermore, some individuals with IOP within the normal range (defined as 10-21 mmHg) develop POAG. It is estimated that nearly 50% of European-derived individuals with open-angle glaucoma have IOP within the normal range and fall under this category while in Asian-derived persons this percentage is even higher.

Since IOP alone cannot predict if someone will develop glaucoma, identification of patient-specific biomarkers within the eye could improve the detection and prevention of POAG damage by identifying the disease onset prior to vision loss symptoms or reducing ongoing damage in those with established POAG. One approach to identify patient-specific biomarkers for POAG is to characterize the mechanical response due to IOP change within the eye, as the geometry and material properties vary in individual eyes. The pilot data presented validate a method for characterizing the in vivo displacement and strain response of the lamina cribrosa (LC) to short-term IOP changes induced by glaucoma medication change.

The optic nerve head (ONH) at the posterior of the eye is comprised of various tissues including the retina, choroid, sclera, prelaminar neural tissue (PLNT), lamina cribrosa (LC), and the optic nerve whose retinal ganglion cells (RGCs) are found in the retina where they receive a light signal. The RGC axons exit the eye posteriorly in bundles running through the pores of the LC and form the optic nerve which carries the light signal to the brain for interpretation.

As a simple biomechanical model, the eye can be thought of as a fluid-filled, pressurized sphere whose walls are mechanically supported by the sclera. The ONH of the eye is subjected to two major loading conditions. First, the ONH is subjected to stress induced by the translaminar pressure gradient. This pressure gradient is defined as the difference between IOP and the tissue pressure within the retrolaminar tissue. Since IOP generally exceeds the retrolaminar pressure, the net stress is caused by IOP; the net compressive stress causes initial damage to RGC axons, and longer term remodeling involving posterior bowing of the ONH called “cupping” or excavation and thinning of the retinal nerve fiber layer (RNFL). Second, the ONH is subjected to a tensile hoop stress carried by the wall of the eye and transmitted by the peripapillary sclera (PPS). This hoop stress is generated by IOP stress acting normal to the cornea and sclera. The deformation due to hoop stress results in widening the ONH in glaucoma. Overall, the IOP-generated strain response is complex, involving both the LC and PPS.

In general, the translaminar pressure gradient caused by an increased level of IOP is expected to produce greater strains in the LC than in the PPS due to the fact that the LC is much thinner than the PPS and the assumption that the LC is less stiff than the PPS. Furthermore, the LC has a less uniform structure compared to the PPS and its pores allow for stress concentrations making it more susceptible to strain. Therefore, the LC represents a weak point within the wall of the eye. Since the LC is especially susceptible to strains and the strains in the LC are linked to detrimental effects such as RGC axon abnormality as well as decreased blood supply, the LC is thought to be the primary site of glaucoma damage.

It is noted that the stiffness of the LC and PPS vary from individual to individual, especially when considering the biomechanical response of the ONH to IOP change. Compliance of the LC for example, is expected to decrease with age. Furthermore, it is not just the stiffness of the LC that mediates the IOP change response but the combined material properties of the LC and adjacent PPS. A stiffer PPS and more compliant LC would be expected to result in bowing of the LC from increased IOP, while a stiffer LC but more compliant PPS, on the other hand, may result in rupture of the LC. If the PPS and LC were both compliant, an increase in IOP may result in elongation of the eye.

Next, to understand the biomechanical response of the ONH to IOP change, it is essential to understand the material properties underlying this complex response. Of particular importance is viscoelasticity. Biological tissues are viscoelastic in nature, meaning their deformation response behaves partly like a viscous fluid and partly like an elastic solid. Viscoelastic materials exhibit three key characteristics: creep, stress-relaxation, and hysteresis. Creep is the slow increase in strain with time when the loading conditions are held constant. Conversely, when an applied strain is constant, viscoelastic materials undergo decreased stress over time. This is known as stress relaxation. Finally, hysteresis occurs when the loading and unloading responses of a material differ due to viscosity. In the current study, the LC was assumed to be a viscoelastic soft tissue, implying its mechanical response is dependent on time.

According to examples of the present disclosure, the mechanical strain response of the LC to IOP change is characterized from medication change, which could be either the initiation of new IOP-lowering medication, or the temporary stopping of medication delivery (to allow IOP to rise). Recent advances in optical coherence tomography (OCT) and computational algorithms, such as digital volume correlation (DVC), have provided researchers with a noninvasive method to measure displacements and strains in biological tissues in vivo. The use of OCT imaging and DVC to measure the in vivo biomechanical response of the LC to IOP change were used to achieve this in the present study.

### 1.4 Optical Coherence Tomography (OCT)

Optical coherence tomography (OCT) is an imaging modality extensively used in ophthalmology to image the layers of the retina and visualize structures of the optic nerve head which may be used for glaucoma diagnostic testing, among other clinical applications. The principle behind OCT is analogous to that of ultrasound imaging, except OCT utilizes light waves instead of sound waves. In OCT imaging, light travels to the tissue sample, is backscattered by the retinal layers and other soft tissue layers of the ONH, then interferes with a light ray sent to a mirror with a known time delay for reference. Using time delay information, the depth profile of a tissue sample can be reconstructed. Consecutive OCT scans may be stacked to form a 3D image volume.

OCT offers a noninvasive and clinically available method to image the layers of the retina and optic nerve head of glaucoma patients in vivo and in situ. OCT uses a broad band light source (typically a supreluminescent diode) as its light source, making it a safe imaging method. Additionally, OCT is fast enough to acquire images in real time. OCT instrumentation primarily consists in two configurations: spectral domain and time domain. Spectral domain OCT (SD-OCT) was used in the present study due to its higher speed and higher resolution compared to time domain OCT (TD-OCT), as well as its increasing use in biomedical applications. Additionally, studies have shown that SD-OCT provides better measurement repeatability compared to TD-OCT.

Digital volume correlation (DVC) is a 3D extension of digital image correlation (DIC), which calculates full displacement and strain fields, and is based on finite element analysis principles. DVC is becoming an increasingly popular method within the biomechanics field for analyzing in vivo tissue deformation. DVC is suitable for correlating images with random, high-contrast speckle or intensity patterns. In the present study, the natural speckle patterns of the LC were visible in OCT scans. Our group and others have demonstrated DVC is a valid method for quantifying strains in OCT scans of the ONH.

DVC estimates displacements from the reference image to the deformed image by correlating groups of pixels in specified correlation windows, also known as subsets. The correlation window contains a unique “signature” from the reference image, which is composed of a central pixel surrounded by neighboring pixels. The size of the correlation window may be specified by the user. First, the algorithm searches for the signature in the deformed image using the specified correlation window. Various locations in the deformed image are checked for possible matches. The possible matches are given a score based on a correlation function which is usually the sum of squared differences of the pixel values. The smallest score (smallest difference) is chosen as the best match. Displacement fields are calculated as the average of pixel displacements contained in the window. From the displacement fields, the resulting strain fields may be derived.

According to examples of the present disclosure, a fast-iterative DVC algorithm can used to calculate IOP change-related strain fields in the LC from medication change. This algorithm allows for iterative refinement of the window size and spacing to optimize speed and accuracy.

The level of IOP remains the most relevant risk factor for glaucoma, however, the level of IOP alone cannot definitively predict if an individual will develop glaucoma. Recent research efforts suggest characterizing the biomechanical response of the eye to IOP change may provide a more effective and patient-tailored approach to assess glaucoma risk. Thus, the goal of the present study was to characterize the short-term biomechanical response of the lamina cribrosa (LC), the primary site of glaucoma damage, to IOP change induced via a change in glaucoma medication, primarily IOP-lowering eye drops. The biomechanical response of the LC to IOP change may be a potential biomarker that could aid in the earlier detection and management of glaucoma.

Previously, in vivo studies have revealed IOP lowering via post trabeculectomy laser suturelysis results in a mean tensile E_{zz} strain in the anterior lamina cribrosa (ALC), implying IOP-lowering treatments provide strain relief to the LC, allowing it to decompress and expand along its axis relative to the reference state at higher IOP. Conversely, when IOP was increased in the eyes of patients wearing tight-fitting swimming goggles, compressive E_{zz} strain was produced. The mean Eᵣᵣ strain was compressive, suggesting that IOP-lowering treatment also provides strain relief in the radial direction allowing the width of the LC, which is often increased due to high level of IOP and seen in excavation, to contract.

According to the present experimental results, it has been found that the LC subjected to IOP-lowering can cause negative or positive ALD change 20 minutes after suturelysis, implying the ALC surface can move anteriorly or posteriorly after IOP lowering. ALD change magnitude was not related to IOP change, however, it was significantly associated with having a lower baseline IOP.

Previous work has also studied how the biomechanical response of the LC to IOP change is related to glaucoma damage. Previous studies have also characterized the in vivo biomechanical strain response of the LC to IOP lowering via suturelysis after a 20 minute duration. The current study seeks to replicate these experiments in a different population consisting of medication-change patients over a longer duration of one week. The overall goal of this study was to establish a method used for characterizing the short-term in vivo biomechanical strain response of the LC to a change in IOP lowering medication. The major aims were: 1) characterize the in vivo ALD change, LC strain response, and strain compliance response to IOP change induced via glaucoma medication change over a period of approximately one week and provide evidence that these measurements are repeatable 2) investigate the relationship of LC strains to IOP change and LC strains to ALD change and 3) investigate the relationship of LC strains and strain compliance to three primary measures of glaucoma damage: RNFL thickness, mean deviation (MD), and visual field index (VFI). To the best of the author's knowledge, this is the first study attempting to measure the short-term (one-week) strain response within the LC using DVC of OCT scans of the ONH before and after IOP change from medication change.

According to examples of the present disclosure, experiments conducted with study participants received a standard diagnostic exam for glaucoma prior to participating in the study. The data included in the present study was collected and analyzed for 23 eyes from 15 patients with POAG undergoing glaucoma medication change. All samples but two were from the eyes of patients starting hypotensive eye drops. One study eye was from a patient stopping eye drops and one eye was from another patient starting oral medication to lower IOP. Patient data was collected in two separate sessions: one pre-treatment and one post-treatment. The mean time between the two sessions was 7.3±1.4 days.

Data was sampled from eyes with varying glaucoma severity, however, the patient demographic for this study was biased towards patients with mild glaucoma damage, defined as mean deviation (MD) greater than −6 dB. The sample population included eyes from males and females of African American, Asian, and Caucasian descent and was biased towards patients of female sex and Caucasian descent. The sample population had an age range of 30 to 80 years, a mean age of 62.1±12.4 years, and a median age of 64 years. (Table 1)

The subjects were divided into two groups. Group 1 consisted of 17 eyes from 12 patients who received IOP-lowering medication and underwent an IOP change in magnitude of at least 4 mmHg after one week. This threshold was chosen based on prior work by our group which demonstrated that the method used in the present study can measure significant strains resulting from IOP changes as small as 2 mmHg. Group 2 consisted of 6 eyes from 5 patients who received IOP-lowering medication and did not undergo a significant IOP change (defined as a change of 0-1 mmHg to allow for error from the tonometer) after the treatment period. No eyes had an IOP change magnitude between 1 mmHg and 4 mmHg.

All experimental study groups consisted of eyes undergoing a change in glaucoma medication. These changes included starting newly prescribed hypotensive eye drops, stopping eye drops, or starting oral medication to lower IOP. Eyes starting a new glaucoma medication were expected to undergo a decrease in IOP after the treatment period of 7.3 days±1.4 days. Strains resulting from IOP decrease were representative of strain relief compared to the reference state. The eyes of patients stopping glaucoma medication were expected to undergo an increase in IOP after one week, representative of increased strain relative to the reference state.

Glaucoma medication-change patient demographics for
entire medication change group and its two subgroups
based on IOP change: Group 1 and Group 2.
There were no eyes with an IOP change between 1 and 4 mmHg.

| | All (Group 1 | Group 1 | Group 2 |
|---|---|---|---|
| | and Group 2) | | |
| Group | All | Medication- | Medication- |
| Description | medication- | change | change eyes |
| | change | eyes | with IOP |
| | eyes | with IOP | change of 0-1 |
| | | change ≥4 | mmHg (“pseudo- |
| | | mmHg | control” group) |
| Sample Size | 23 eyes from 15 | 17 eyes from 12 | 6 eyes from |
| | patients | patients | 5 patients |
| Age (years) | 62.1 ± 12.4 | 62.2 ± 13.7 | 61.8 ± 9.0 |
| Sex | 15 F, 8 M | 11 F, 6 M | 4 F, 2 M |
| Medication | 7.3 ± 1.4 | 7.3 ± 1.4 | 7.5 ± 1.2 |
| Change Period | | | |
| (days) | | | |
| Baseline IOP | 18.3 ± 6.6 | 20.3 ± 5.8 | 12.5 ± 5.2 |
| (mmHg) | | | |
| IOP Change | −4.3 ± 2.8 | −5.8 ± 1.5 | −0.3 ± 0.8 |
| (mmHg) | | | |
| MD (dB) | −6.0 ± 5.7 | −6.6 ± 5.8 | −4.3 ± 5.8 |
| VFI | 0.86 ± 0.13 | 0.85 ± 0.13 | 0.90 ± 0.14 |
| Average RNFL | 66.6 ± 8.0 | 65.2 ± 7.3 | 70.7 ± 9.1 |
| (μm) | | | |

Intraocular pressure (IOP) was measured in each eye using a rebound tonometer (iCare TA01i, iCare Finland Oy, Espoo, Finland). The iCare tonometer was used to measure IOP in this study due to its high accuracy and high reproducibility comparable to that of Goldman applanation tonometry. The iCare tonometer used in the study had an accuracy of ±1.2 mmHg (20 mmHg) and ±2.2 mmHg (>20 mmHg) for a 95% tolerance interval relative to manometry and repeatability (coefficient of variation) less than 8. In addition to providing accurate and consistent measurements, the iCare tonometer was selected for convenience as it does not require a local anesthetic prior to use nor specialized training to use.

IOP for each study eye was measured prior to each imaging session. Baseline or pre-treatment IOP and post-treatment IOP were each recorded as the mean of six consecutive measurements per eye. Repeated measurements were taken to determine the average IOP in each eye until the tonometer screen displayed a low or no variability mean IOP reading. An average IOP reading with low variability indicated the standard deviation of the six consecutive measurements was higher than normal but was considered acceptable, as low variability is unlikely to impact the result. If the initial mean IOP reading indicated high variability, the average of two no or low variability means was recorded as the final IOP measurement.

All IOP measurements were taken with patients sitting in an upright position and without use of a local anesthetic. Patients were directed to look forward such that the chin was parallel to the ground. Measurements were taken with the rebound tonometer probe positioned perpendicularly to the surface of the central cornea with the tip of the probe approximately 4 to 8 mm away.

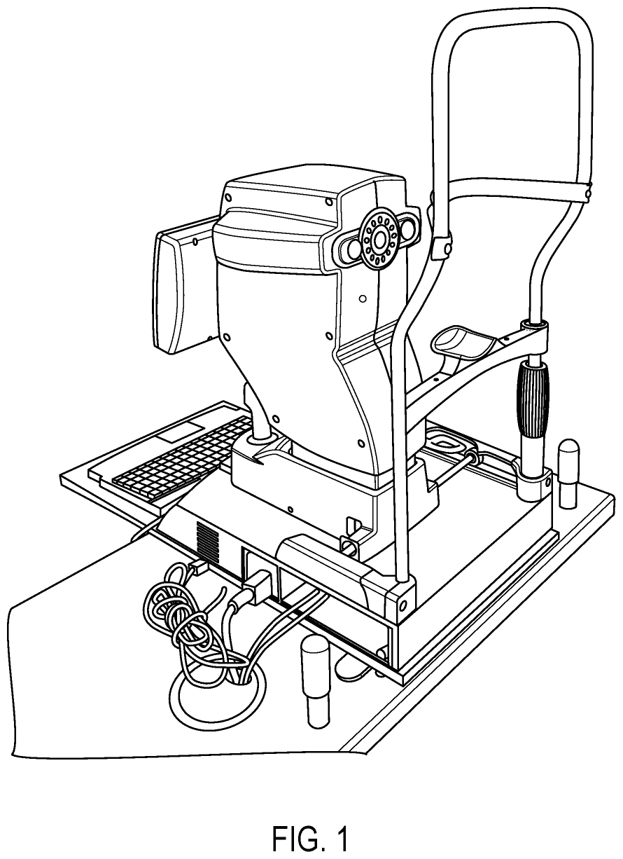

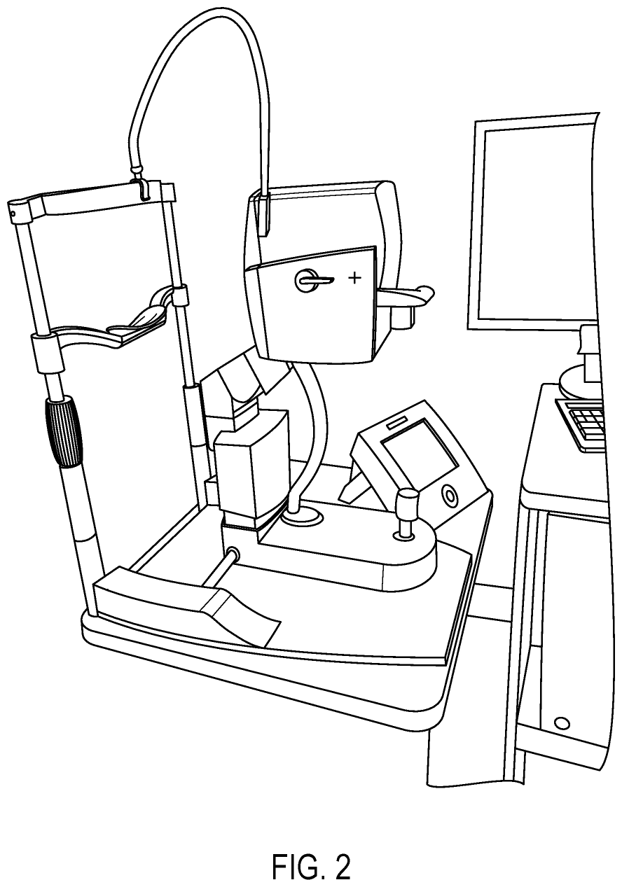

FIG. 1 shows an IOL Master, a standard instrument widely used in ophthalmic clinics, was used to measure the axial length and curvature of each eye.

In order to prepare for optical coherence tomography imaging, the average axial length and corneal curvature of each eye were measured using an IOL Master (Carl Zeiss Meditec, Inc., Dublin, CA, USA) (FIG. 1), an ocular biometry instrument based on partial coherence interferometry (PCI), a non-contact method routinely used in ophthalmology that offers high resolution and high reproducibility. Axial length measurement was taken as the average of five subsequent measurements. In order to calibrate the OCT instrument's optical magnification for each eye, corneal curvature was taken as an average of the maximum and minimum measurements at baseline IOP and at IOP post-treatment.

Optical coherence tomography (OCT) is an imaging modality extensively used in ophthalmology to image the layers of the retina and visualize structures of the optic nerve head which may be used for glaucoma diagnostic testing, among other clinical applications. The principle behind OCT is analogous to that of ultrasound imaging, except OCT utilizes light waves instead of sound waves.

In OCT imaging, light travels to the tissue sample, is backscattered by the retinal layers and other soft tissue layers of the ONH, then interferes with a light ray sent to a mirror with a known time delay for reference. Using time delay information, the depth profile the reflected light relative to echo delay can be reconstructed. This occurs at a single point and is known as an amplitude scan or A-scan. Scanning a sample laterally at multiple points results in a grayscale cross-sectional image, known as a B-scan (brightness scan), where the grayscale value at each point represents the brightness amplitude of the reflected light. Consecutive OCT scans may be stacked to form a 3D image volume. The present study acquired B-scans spaced at equal intervals circumferentially around the optic nerve head. This resulted in 2D cross-sectional images in which the height of the scan represented the axial direction, and the length of the scan represented the radial direction. These are known as radial B-scans.

The optic nerve head (ONH) of each study eye was imaged using a Spectralis (Heidelberg Engineering, Heidelberg, Germany) (FIG. 2) at the baseline IOP and at IOP post treatment. Patients were imaged in a seated position with the chin rest adjusted so that the patient's chin was parallel to the ground. After turning off the lights, patients were directed to look forward, focusing on a target light, and blink several times before image acquisition. Images were acquired with the OCT instrument set to high resolution and auto-brightness mode. To optimize image quality and visibility of the LC, the OCT was adjusted such that the brightness of the visible tissue was as high as possible, and the tissue cross section was positioned such that it occupied the lower two-thirds of the window. During each imaging session, three consecutive image volumes were taken for each eye approximately 30 seconds apart. After the first volume was acquired, the subsequent volumes were automatically oriented by the Heidelberg software registration.

FIG. 2 shows a Heidelberg Spectralis was used at each imaging session to acquire radial OCT image volumes consisting of 24 slices per volume.

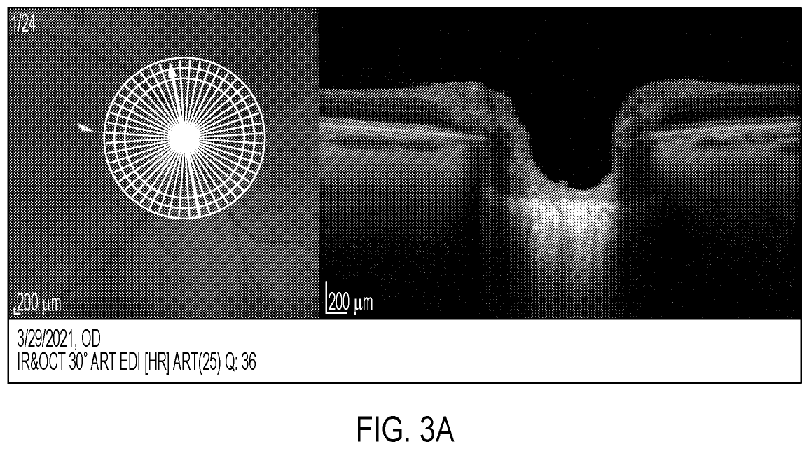

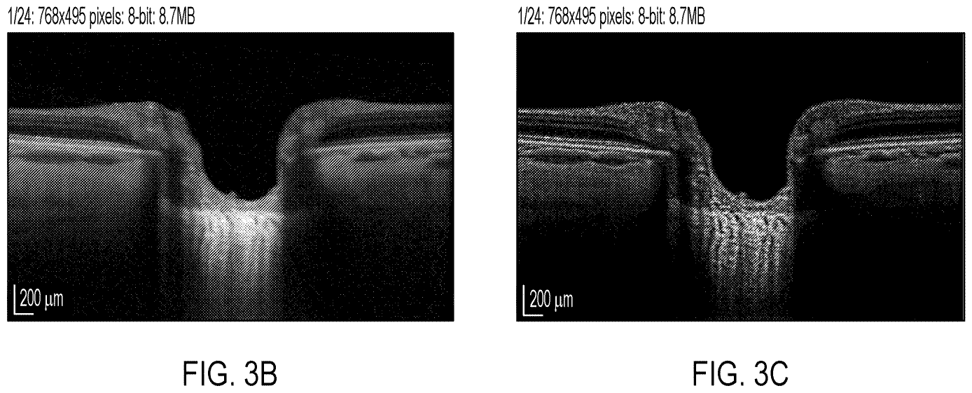

FIG. 3A, FIG. 3B, FIG. 3C show radial OCT scans of the ONH were acquired using the Heidelberg OCT. Each OCT image volume consisted of 24 evenly spaced slices, where FIG. 3A shows an original scan and orientation map, FIG. 3B shows an original OCT image after cropping in Matlab, and FIG. 3C shows an image processed with CLAHE and a gamma filter to enhance contrast of the speckle pattern to optimize DVC correlation.

Each image volume consisted of 24 radial B-scans (optical cross-sections) centered about the ONH (FIG. 3A, FIG. 3B, and FIG. 3C). Circumferential scans were taken in 7.5° clockwise steps with the first scan oriented perpendicular to the axis connecting the center of the Bruch's membrane opening and the fovea. The resolution of each scan was 768×495 in the (R, Z) plane and had a resolution in the Z direction (anterior-posterior) of 3.87 μm/pixel. Resolution in the R (radial) direction ranged from 5.95±0.29 (5.66-6.24) μm/pixel. Resolution in the R-direction varied among each eye due to the optical magnification used to adjust for patient-specific corneal curvature of each eye. The R-scaling factor for each eye determined by the Heidelberg software was recorded for DVC analysis.

Radial OCT scans were pre-processed using MATLAB (R2019a, Mathworks, Natick, MA, US). The 24 de-identified OCT scans for each volume were cropped to render 768×495-pixel cross-sectional images of the retina. Images were then converted into 8-bit intensity-valued pixels. Each image was trimmed of 13 pixels on the left and right edges to reduce errors associated with DVC calculation along image borders. Additionally, 35 pixels were cropped from the bottom (posterior) image border to remove overlaid scale bars, rendering 642×460-pixel images. Furthermore, each scan was vertically cropped about the horizontal midpoint such that R=0 corresponded to the center of the ONH in each image and Z=0 corresponded to the top (anterior) border of the image. The circumferential direction, or θ-direction, was oriented such that θ=0° corresponded to the inferior-superior cross-section. This created a set of 371×360×48 images per image volume spaced 7.5° apart circumferentially about the ONH.

All image volumes were filtered and contrast-enhanced in ImageJ Fiji (FIG. 3), using the software's contrast-limited adaptive histogram equalization (CLAHE) algorithm, a modified version of adaptive histogram equalization (AHE) designed to minimize noise amplification. It has been demonstrated that CLAHE contrast enhancement can improve DVC correlation. Within CLAHE, the block size was set to 14, the histogram bins were set to 256, the maximum slope was set to 3.5, and the mask was set to “None” type. In addition to CLAHE, a gamma filter with a correction value of 1.75 was used to preserve tissue speckle patterns within each image to prevent filtering out fine tissue details as noise. Preserving the unique speckle pattern within each image of the ONH tissue is crucial for optimizing DVC correlation.

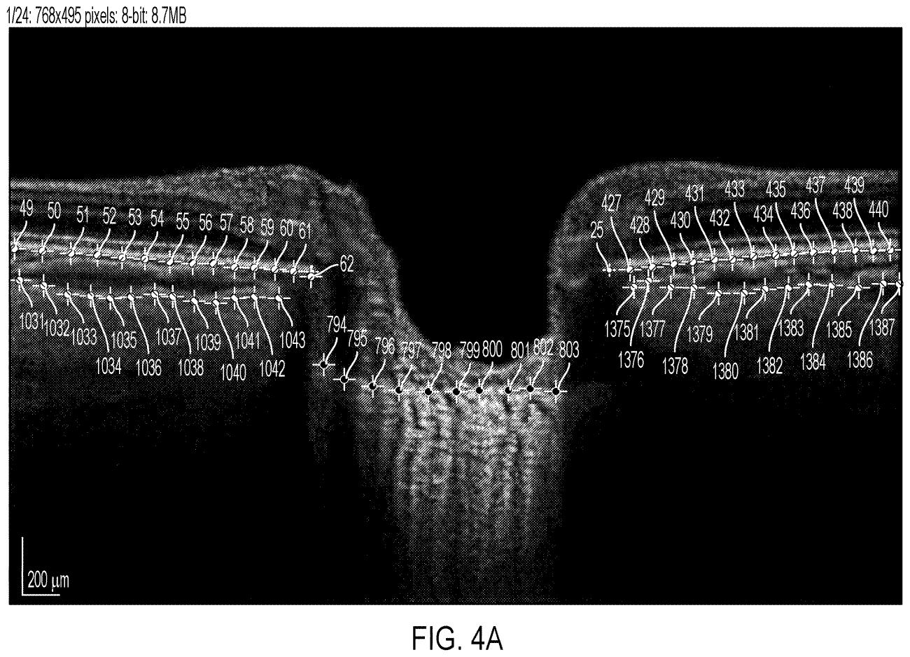

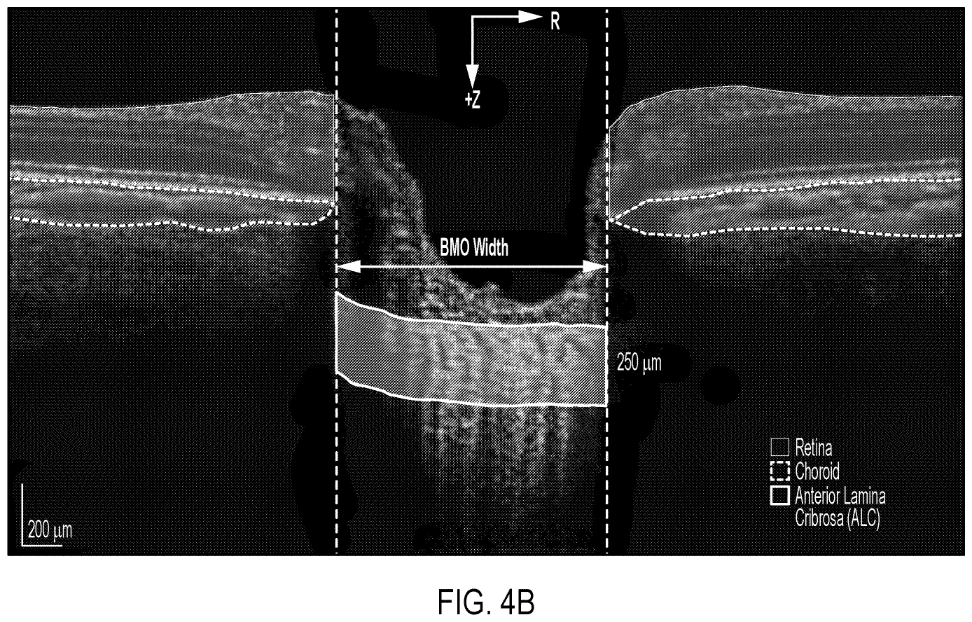

FIG. 4A and FIG. 4B shows an optic nerve head in each of the 24 slices in every OCT volume was segmented by hand in ImageJ to define the boundaries of the ALC, retina, choroid, and sclera. The posterior border of the ALC was segmented automatically in MATLAB by drawing a border 250 μm below the anterior border of the ALC. The pixel coordinates were imported into MATLAB for DVC post-processing analysis.

In preparation for DVC, the filtered image volume acquired at baseline IOP with the greatest focus and least noise was selected for each eye. Several soft tissue layers of the ONH in each scan were segmented by hand in ImageJ Fiji (V.H.) and verified by an ophthalmologist (H.A.Q.) (FIG. 4A). For each scan, the Bruch's membrane opening (BMO), the posterior border of Bruch's membrane, the choroid-sclera interface, and the anterior LC border were segmented, and each structure's pixel coordinates (R, Z, θ) were saved for DVC analysis. The BMO was marked twice per scan and a horizontal line was drawn between the two points to define the BMO width. The Bruch's membrane, choroid-sclera interface, and ALC border were marked without a set number of points such that the points were spaced approximately every 15-25 pixels. The ALC border was only marked within the BMO width. Anterior LC depth (ALD) was defined as the vertical distance between the BMO width and the ALC border. The left and right Bruch's membranes and the left and right choroid sclera interfaces were segmented in order to obtain DVC-calculated strains in the retina, choroid and sclera.

After importing the pixel coordinates of each hand-segmented boundary, the ALC was segmented from the posterior lamina cribrosa (PLC) by drawing a border 250 μm posterior to the ALC in MATLAB. The ALC-PLC border was not segmented manually since the PLC border cannot be distinguished visually in OCT scans. The ALC region was defined with a thickness of 250 μm based on the average histological thickness for the human ALC and was additionally based upon the previous method employed by our group for estimating in vivo strains in the human LC.

DVC estimates displacements from the reference image to the deformed image by correlating groups of pixels in specified correlation windows, also known as subsets. The correlation window contains a unique “signature” from the reference image which is composed of a central pixel surrounded by neighboring pixels. The size of the correlation window may be specified by the user. First, the algorithm searches for the signature in the deformed image using the specified correlation window. Various locations in the deformed image are checked for possible matches. The possible matches are given a score based on a correlation function which is usually the sum of squared differences of the pixel values. The smallest score (smallest difference) is chosen as the best match. Displacement fields are calculated as the average of pixel displacements contained in the window. From the displacement fields, the resulting strain fields may be derived.

In the current study, a fast-iterative DVC algorithm was used to calculate IOP change-related strain fields in the LC from medication change. This algorithm, used in previous work by our group, allowed for iterative refinement of the window size and spacing to optimize speed and accuracy. The fast-iterative DVC algorithm developed by the Bar-Kochba group was used to compute displacement fields and the corresponding strain fields as a result of IOP change from glaucoma medication change. Prior to performing DVC in MATLAB, the radial scaling factor (μm/pixel) and whether the eye was a left eye or right eye were specified. The DVC correlation coefficient was held constant at 0.055 and the best reference image, second best reference image, and best post-treatment image were designated within the code. The (R, Z, θ) pixel coordinates for each hand-segmented ONH region were imported into the code to define the regional boundaries for DVC post-processing. This allowed for obtaining the average strain within the ALC, retina, choroid, and sclera.

Displacements in the R, Z, and θ direction were calculated by applying DVC to the undeformed reference image (image taken at baseline IOP) and the deformed image (image taken at post-treatment IOP). The pixel displacements were later converted to μm displacements using the recorded scaling factors. Displacement gradients with respect to R and Z for locally defined pixel neighborhoods within the R-Z plane were determined by local displacement fitting. Displacement gradients with respect to the θ-direction were determined by 4th order polynomial fitting for displacements at every in-plane (R,Z) pixel for all image volume slices. To ensure boundary continuity, polynomial fitting for displacements was repeated twice for image slices oriented at θ=0 and θ=2π.

From the displacements, the strain tensor was calculated in cylindrical coordinates using the standard Green-Lagrange strain equations (Eqs. 1-6).

$${E}_{rr} = \frac{\partial {U}_{r}}{\partial r} + \frac{1}{2} ( {( \frac{\partial {U}_{r}}{\partial r} )}^{2} + {( \frac{\partial {U}_{θ}}{\partial r} )}^{2} + {( \frac{\partial {U}_{z}}{\partial r} )}^{2} ) \qquad ( 1 )$$

$${E}_{zz} = \frac{\partial {U}_{z}}{\partial z} + \frac{1}{2} ( {( \frac{\partial {U}_{r}}{\partial z} )}^{2} + {( \frac{\partial {U}_{θ}}{\partial z} )}^{2} + {( \frac{\partial {U}_{z}}{\partial z} )}^{2} ) \qquad ( 2 )$$

$${E}_{θθ} = \frac{{U}_{r}}{r} + \frac{1}{r} \frac{\partial {U}_{θ}}{\partial θ} + \frac{1}{2} ( \frac{1}{{r}^{2}} {( \frac{\partial {U}_{z}}{\partial θ} )}^{2} + {( \frac{1}{r} \frac{\partial {U}_{r}}{\partial θ} - \frac{{U}_{θ}}{r} )}^{2} + {( \frac{1}{r} \frac{\partial {U}_{θ}}{\partial θ} + \frac{{U}_{r}}{r} )}^{2} ) \qquad ( 3 )$$

$${E}_{rz} = \frac{1}{2} ( \frac{\partial {U}_{r}}{\partial z} + \frac{\partial {U}_{z}}{\partial r} + \frac{\partial {U}_{z}}{\partial r} \frac{\partial {U}_{z}}{\partial z} + \frac{\partial {U}_{θ}}{\partial r} \frac{\partial {U}_{θ}}{\partial z} + \frac{\partial {U}_{z}}{\partial r} \frac{\partial {U}_{z}}{\partial z} ) \qquad ( 4 )$$

$${E}_{θr} = \frac{1}{2} ( \frac{\partial {U}_{θ}}{\partial r} + \\ \frac{1}{r} ( \frac{\partial {U}_{r}}{\partial θ} + \frac{\partial {U}_{r}}{\partial θ} \frac{\partial {U}_{r}}{\partial r} + \frac{\partial {U}_{z}}{\partial θ} \frac{\partial {U}_{z}}{\partial r} + \frac{\partial {U}_{θ}}{\partial θ} \frac{\partial {U}_{θ}}{\partial r} + {U}_{r} \frac{\partial {U}_{θ}}{\partial r} - {U}_{θ} \frac{\partial {U}_{r}}{\partial r} - {U}_{θ} ) ) \qquad ( 5 )$$

$${E}_{θz} = \frac{1}{2} ( \frac{\partial {U}_{θ}}{\partial z} + \frac{1}{r} ( \frac{\partial {U}_{z}}{\partial θ} + \frac{\partial {U}_{z}}{\partial θ} \frac{\partial {U}_{z}}{\partial z} + \frac{\partial {U}_{r}}{\partial θ} \frac{\partial {U}_{r}}{\partial z} + \frac{\partial {U}_{θ}}{\partial θ} \frac{\partial {U}_{θ}}{\partial z} + {U}_{r} \frac{\partial {U}_{θ}}{\partial z} - {U}_{θ} \frac{\partial {U}_{r}}{\partial z} ) ) \qquad ( 6 )$$

From the 2D strain tensor (Eᵣᵣ, E_{zz}, E_{rz}), maximum principal strain, Eₘₐₓ, and maximum shear strain, Γₘₐₓ, were determined for strains contained in the R-Z plane, the plane imaged by the OCT radial scans (Eqs. 7 and 8). Maximum principal strains were not calculated using the 3D strain tensor due to displacement resolution in the θ-direction growing poorer as radial distance increased.

$${E}_{max} = \frac{{E}_{r r} + {E}_{z z}}{2} + \sqrt{{( \frac{{E}_{r r} - {E}_{z z}}{2} )}^{2} + {E}_{r z}^{2}} \qquad ( 7 )$$

$${Γ}_{max} = \sqrt{{( \frac{{E}_{r r} - {E}_{z z}}{2} )}^{2} + {E}_{r z}^{2}} \qquad ( 8 )$$

Strain correlation error was determined by taking the difference between the applied strain and the observed strain determined by DVC. The observed strain was calculated by duplicating the reference image volume (image at baseline IOP) and warping it with a known stretch value, then calculating the resulting displacement and strain fields using DVC. Based on previous methods by our group, the warped reference image was created by applying a rigid body translation of 10 μm in the Z-direction followed by a uniform 2% tensile strain in the R-direction and 2% compressive strain in the Z-direction. To account for error due to eye movement and blood vessel pulsation, strain baseline error was calculated by taking the difference between strain calculated in DVC for duplicate image volumes (two image volumes taken 30 seconds apart) for the same eye at baseline IOP.

The DVC results were post-processed within the MATLAB code to minimize strain error resulting from displacement correlation error. A DVC correlation filter was used to remove areas with poor displacement correlation, reduce displacement errors along image borders, and remove displacements with average absolute displacement error greater than 0.25 pixel by using threshold and subset size settings previously determined by our group. Additionally, the filter was designed to minimize calculating displacements outside of tissue boundaries.

Regions containing correlation coefficients less than the threshold of 0.055 were considered to have poor contrast that may increase correlation error and were removed by the DVC correlation filter. To reduce displacement correlation errors along boundaries containing the tissue sample, displacement calculations were excluded if they fell within 25 pixels of the bottom (posterior) image border or within 32 pixels from the left or right image border. DVC often results in higher correlation errors along boundaries due to discontinuities; excluding the boundaries from DVC analysis is a common practice. Field discontinuities within the target analysis area can further increase correlation error. Thus, displacements fields and discontinuities in the R-Z plane were smoothed using a 2D Gaussian filter developed by our group. Out-of-plane displacements corresponding to the circumferential or θ-direction were smoothed using a 1D Gaussian filter for each image slice at each in-plane (R, Z) pixel. The Gaussian smoothing filters were designed to reduce spikes in the displacement field while maintaining the average gradient. Finally, in-plane displacement outliers were filtered by removing Ur and Uz displacements exceeding 5 μm or exceeding the average displacement within a locally defined neighborhood by 10 μm.

All data reported in the current study is represented as mean±standard deviation. Recall, the study consists of n=23 eyes from 15 patients for the entire medication-change group (Group 1 and Group 2), n=17 eyes from 12 patients for subgroup Group 1, and n=6 eyes from five patients for subgroup Group 2 unless otherwise specified. For all statistical analyses, a p-value of 0.05 or less was considered significant and a p-value between 0.05 and 0.10 was considered borderline significant.

To determine whether DVC-measured strains were significantly different from zero, mean strains were compared to zero using a paired T-test. Paired-sample T-tests were also used to determine if mean strains exceeded their corresponding mean baseline error and mean correlation error. Linear regression analysis was used to test relationships between strains, IOP change, IOP percent change, ALD change, damage, and age. For all statistical tests conducted in the current study, normal distribution of the data was assumed. The statistical analyses in the current study did not account for inter-eye correlation effects.

Data was originally collected for a total of 43 eyes (35 medication-change eyes and 8 control eyes) from consenting patients, however only data from 23 medication change eyes from 15 patients were included in the study analysis. A total of 19 eyes from 13 patients (12 medication-change eyes and 7 control eyes) were excluded from analysis primarily due to poor image quality or poor visibility of the ALC, which in some samples was nearly entirely obscured by shadows from the overlying central blood vessels. The remaining control eye from an additional patient was excluded from the study since group outcomes cannot be compared if the control group only has one sample. In total, 20 eyes from 14 patients were excluded.

After the treatment period, IOP decreased modestly and the mean IOP change was −4.4±2.8 mmHg for the entire medication change group (n=XX). The mean IOP change for Group 1 was −5.8±1.5 mmHg while the mean IOP change for Group 2 was −0.3±0.8 mmHg. Group 2 was considered a control group with minimal pressure change. The entire medication change group had a percent IOP change of 22.6%±14.5%, Group 1 had an IOP percent change of 29.6%±8.2%, and Group 2 had an IOP percent change of 2.7%±8.1%.

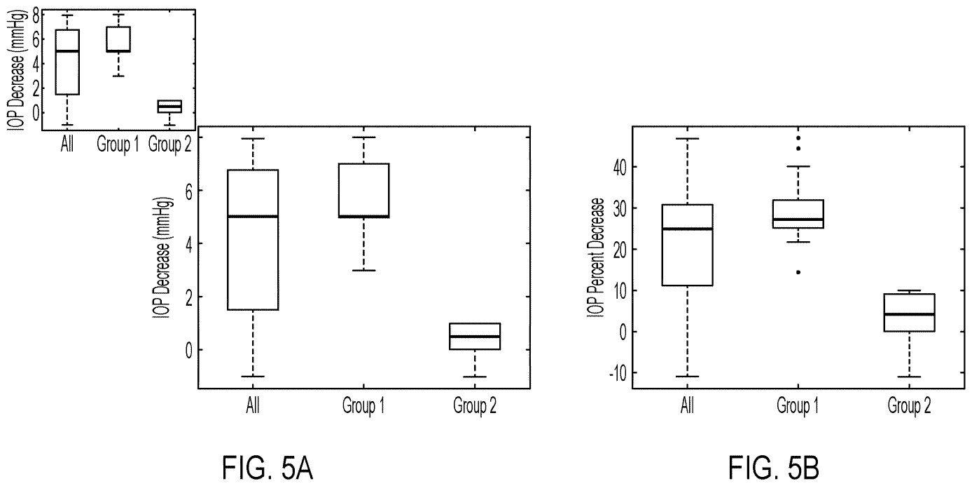

FIG. 5A and FIG. 5B show plots of IOP decrease for each test group where after an average of one week of starting glaucoma medication (hypotensive eye drops), IOP decreased in some but not all medication change eyes. The entire medication change group was subdivided into two groups based on IOP decrease magnitude. Group 1 contained eyes with an IOP change of at least 4 mmHg while Group 2 contained eyes whose IOP did not change after the treatment period (defined as an IOP change of 0-1 mmHg). No eyes underwent an IOP change between 1 and 4 mmHg. FIG. 5A shows a plot of IOP decrease magnitude and FIG. 5B shows a plot of IOP percent decrease.

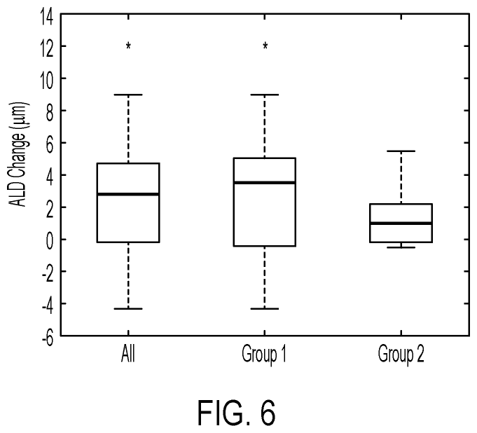

FIG. 6 shows plots of ALD Change (μm) after IOP change induced via glaucoma medication change was calculated using DVC. A positive ALD change represents posterior displacement of the ALC border while a negative ALD change represents anterior displacement. An asterisk indicates the group mean was significantly different from zero (p-value less than or equal to 0.05). The median ALD change was positive for all groups and was significant for the entire medication change group and Group 1, suggesting on average the ALC border moved posteriorly after IOP change over the course of one week.

The ALD displacement for all experimental groups was positive on average after IOP lowering (FIG. 6). A positive ALD change was defined as posterior displacement of the ALC border (moving away from the front of the eye), while a negative ALD change was defined as anterior displacement of the ALC border (moving towards the front of the eye). The ALD displacement for the entire medication change group was 2.32±3.40 μm (p=0.004), suggesting the ALC border moved posteriorly on average after IOP decrease. However, as suggested by the high variability denoted by the standard deviation, the ALC border moved posteriorly in some cases and anteriorly in others. ALD displacement was posterior on average at 2.61±3.73 μm (p=0.011) for Group 1 and was 1.50±2.27 μm for Group 2 but was not significantly different from zero.

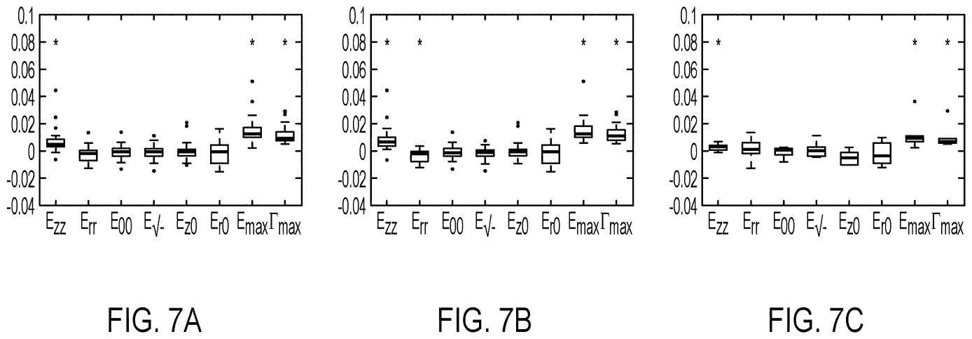

FIG. 7A, FIG. 7B, and FIG. 7C show plots of ALC strains as a result of medication-change induced IOP change for each group. An asterisk (*) indicates p-value less than or equal to 0.05. FIG. 7A shows a plot for the entire medication change group, FIG. 7B shows a plot for Group 1, and FIG. 7C shows a plot for Group 2. IOP decrease produced by hypotensive eye drops resulted in significant tensile E_{zz} and compressive Eᵣᵣ for Group 1. For Group 2, E_{zz} was relatively small compared to E_{zz} of Group 1 and Eᵣᵣ was not significant.

The distribution of the ALC strain response to IOP change across the entire medication change group, Group 1, and Group 2 was plotted in MATLAB for visualization (FIG. 7A, FIG. 7B, and FIG. 7C) and the mean strains are reported in (Table 2). IOP decrease (−4.4±2.8 mmHg) induced via glaucoma medication change resulted in a tensile mean E_{zz} strain of 0.8%=1.0% (p=0.0004) that exceeded baseline error (p=0.00314) for the entire medication change group, indicating the ALC on average expanded in the Z or axial direction (anterior-posterior) relative to the reference state. The IOP decrease also resulted in a compressive Eᵣᵣ strain of −0.2%+0.6% (p=0.125), suggesting the ALC contracted in the radial direction after pressure lowering; however, this strain was not significantly different from zero and did not exceed baseline or correlation error for the entire medication change group. The maximum principal strain, Eₘₐₓ, was 1.6%+1.1% and the maximum shear strain, Γₘₐₓ, was 1.3%±0.7% (p=5.03E-7 and p=4.24E-8, respectively).

For Group 1, IOP decrease (−5.8±1.5 mmHg) resulted in tensile mean E_{zz} (E_{zz}=1.0%±1.1%; p=0.003) and significant compressive mean Eᵣᵣ (Eᵣᵣ=−0.3%±0.5%; p=0.012). Group 1 also had significant Eₘₐₓ (Eₘₐₓ=1.7%±1.0%; p=5.35E-6) and Γₘₐₓ (Γₘₐₓ=1.4%±0.7%; p=4.33E-9). E_{zz} exceeded baseline error (p=0.00532) and Eᵣᵣ exceeded both baseline and correlation error (p=0.0225 and p=0.0309, respectively).

For Group 2, negligible IOP change (−0.3±0.8 mmHg) resulted in a significant Eₘₐₓ, Γₘₐₓ, and tensile E_{zz} (p=0.053, p=0.041, and p=0.054, respectively). The average E_{zz} for Group 2 was 0.3%±0.3% and was borderline greater than baseline error (p=7.52E-2). The mean E_{zz} for Group 2 was about three times smaller than the mean E_{zz} for Group 1.

Table 2. Strains and ALD change in the ALC following IOP change were quantified using DVC for the entire medication change group, Group 1, and Group 2. IOP lowering from starting glaucoma eye drops resulted in a positive (tensile) E_{zz} strain for all groups, indicating the LC expanded along the Z-direction (axial direction or anterior-posterior direction). However, the magnitude of E_{zz} for the eyes that did not undergo significant IOP change in Group 2, was approximately three times smaller than that of Group 1. Eᵣᵣ was negative (compressive) and significant for Goup 1, demonstrating the LC contracts in the radial direction following IOP lowering from eye drops. On average, ALD change was positive and significant for Group 1 and the entire medication change group indicating that the LC surface migrated posteriorly.

TABLE 1
Strains and ALD change in the ALC after medication change IOP
change for entire medication change group, Group 1, and Group 2.

| | All | Group 1 | Group 2 |
|---|---|---|---|
| Ezz | 0.008 ± 0.010 | 0.010 ± 0.011 | 0.003 ± 0.003 |
| | (p = 0.001) | (p = 0.003) | (p = 0.054) |
| Err | −0.002 ± 0.006 | −0.003 ± 0.005 | 0.001 ± 0.009 |
| | (p = 0.125) | (p = 0.012) | (p = 0.762) |
| Ett | −0.001 ± 0.008 | −0.001 ± 0.009 | −0.002 ± 0.009 |
| | (p = 0.516) | (p = 0.699) | (p = 0.762) |
| Ert | −0.001 ± 0.012 | 0.002 ± 0.008 | −0.011 ± 0.017 |
| | (p = 0.560) | (p = 0.354) | (p = 0.196) |
| Etz | −0.001 ± 0.005 | −0.001 ± 0.005 | 0.001 ± 0.006 |
| | (p = 0.553) | (p = 0.323) | (p = 0.653) |
| Erz | −0.001 ± 0.005 | 0.000 ± 0.006 | −0.001 ± 0.004 |
| | (p = 0.637) | (p = 0.770) | (p = 0.626) |
| Emax | 0.016 ± 0.011 | 0.017 ± 0.010 | 0.013 ± 0.012 |
| | (p = 0.000) | (p = 0.000) | (p = 0.053) |
| Gmax | 0.013 ± 0.007 | 0.014 ± 0.007 | 0.011 ± 0.009 |
| | (p = 0.000) | (p = 0.000) | (p = 0.041) |
| ALD | 2.318 ± 3.400 | 2.607 ± 3.734 | 1.498 ± 2.269 |
| (μm) | (p = 0.004) | (p = 0.011) | (p = 0.167) |

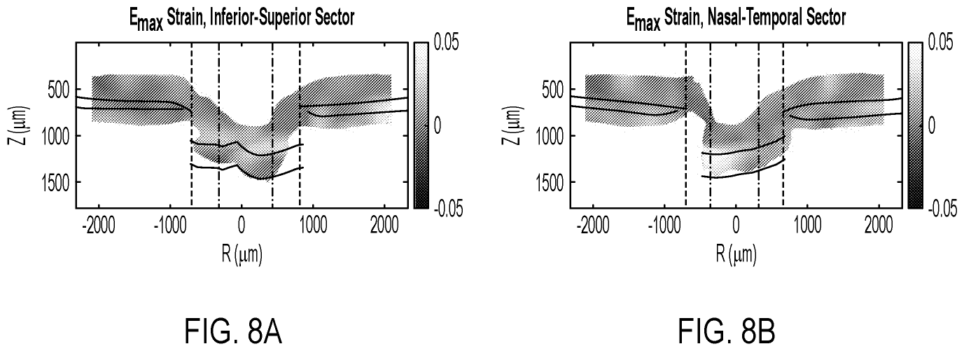

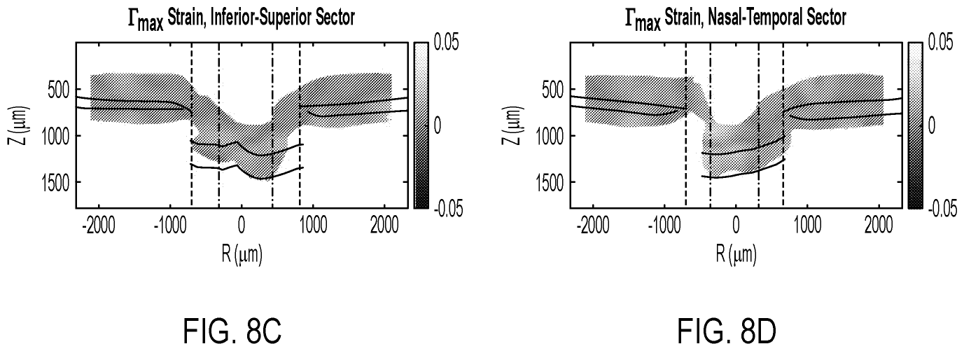

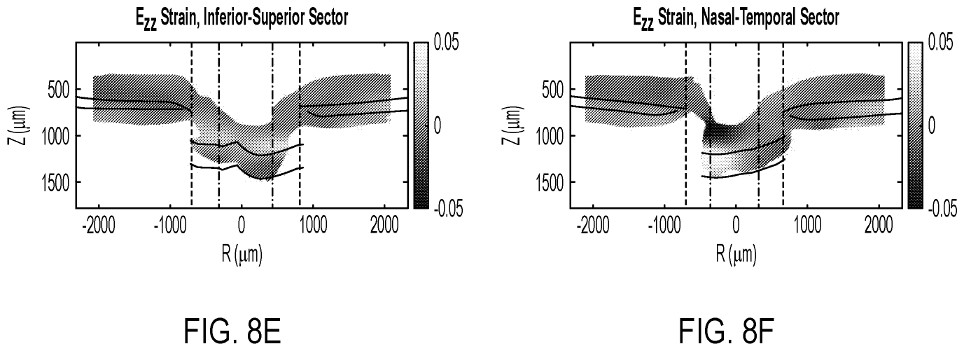

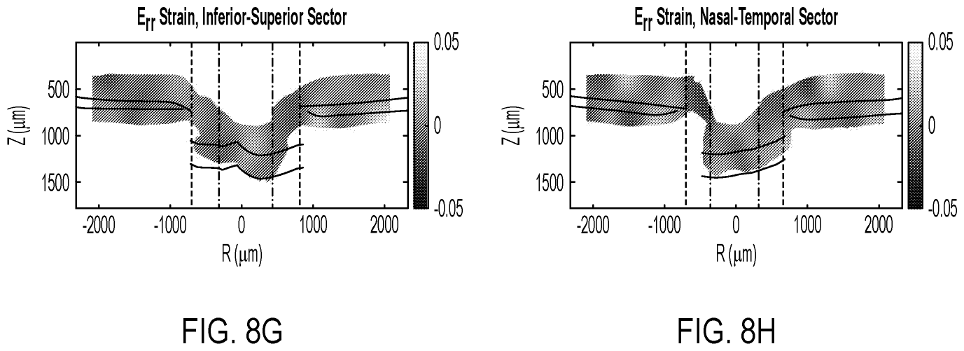

FIG. 8A, FIG. 8B, FIG. 8C, FIG. 8D, FIG. 8E, FIG. 8F, FIG. 8G, and FIG. 8H show plots of DVC strain maps of the ONH after IOP change from medication change were generated in MATLAB. These sample strain maps were taken from a Group 1 eye with an IOP decrease of −7 mmHg and ALC % correlation of 64%. FIG. 8A shows Eₘₐₓ strain map for inferior-superior slice FIG. 8B shows Eₘₐₓ strain map for nasal-temporal slice FIG. 8C shows Γₘₐₓ strain map for inferior-superior slice FIG. 8D shows Γmax strain map for nasal-temporal slice FIG. 8E shows E_{zz} strain map for inferior-superior slice FIG. 8F shows E_{zz} strain map for nasal-temporal slice FIG. 8G shows Eᵣᵣ strain map for inferior-superior slice FIG. 8H shows Eᵣᵣ strain map for nasal-temporal slice.

For all the entire medication change group, Group 1, and Group 2, there were no significant differences in mean E_{zz} or mean Eₘₐₓ within the central ALC compared to the peripheral ALC. Furthermore, ANOVA indicated there were no differences in mean E_{zz}, mean Eₘₐₓ, or mean ALD change by quadrant of the LC. The average RNFL thickness was 66.61±7.99 μm for the entire medication change group, however, RNFL varied significantly by LC quadrant (p=3.27E-12). RNFL was 76.74±11.35 μm in the inferior quadrant, 80.09±15.38 μm in the superior quadrant, 61.13±12.09 μm in the nasal quadrant, and 48.74±14.13 μm in the temporal quadrant. RNFL thickness was greater in the inferior quadrant than in the nasal and temporal quadrants (p=1.1E-3 and p=0.0000, respectively). Additionally, RNFL thickness was greater in the superior quadrant than in the nasal and temporal quadrants (p=1E-4 and p=0.0000, respectively). Finally, RNFL thickness was greater within the nasal quadrant compared to the temporal quadrant (p=1.36E-2).

Linear regression was used to assess whether IOP decrease magnitude was significantly related to strain magnitude. For the entire medication-change group, there was a borderline negative association between Eᵣᵣ and IOP decrease (R²=0.156, p=0.062), where Eᵣᵣ becomes more negative or compressive with increased IOP decrease. Additionally, there was a borderline positive association between Gₘₐₓ and IOP decrease (R²=0.125, p=0.098), where Γₘₐₓ increases in magnitude with increased IOP decrease. There were no significant or borderline significant associations between the other unique components of the strain tensor—E_{zz}, E_{rz}, E_{zt}, and Eᵣₜ—with IOP decrease. Furthermore, neither Eₘₐₓ nor ALD change was associated with IOP decrease.

In Group 1, greater Γₘₐₓ was associated with a greater IOP decrease (R²=0.313 p=0.019). Unlike the entire medication-change group, there was a borderline positive correlation between Eₘₐₓ and IOP decrease (R²=0.192, p=0.079) but no association between Eᵣᵣ and IOP decrease. For Group 1, there were no associations between the other strain components and IOP decrease, nor was there an association between ALD change and IOP decrease.

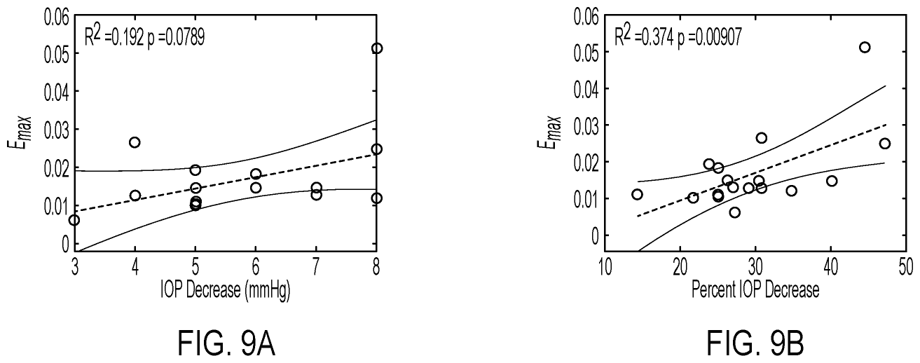

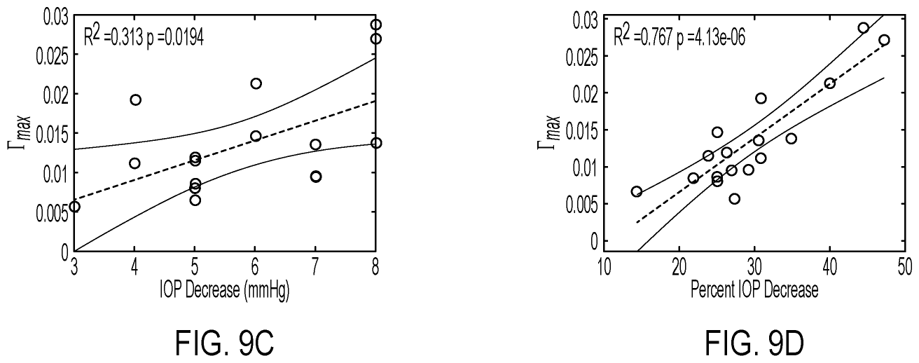

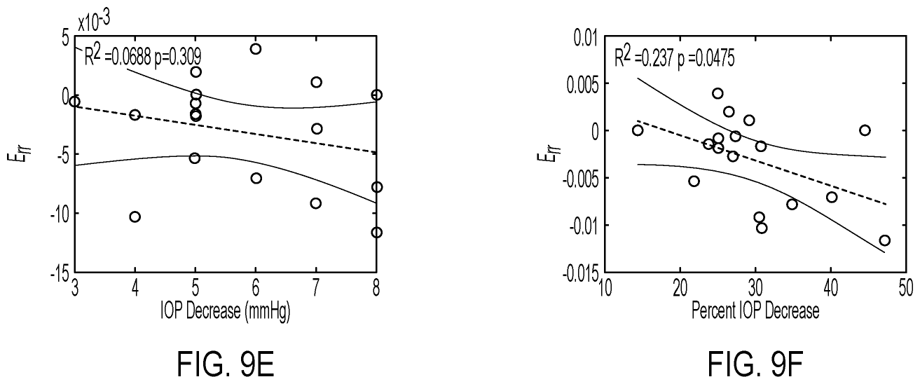

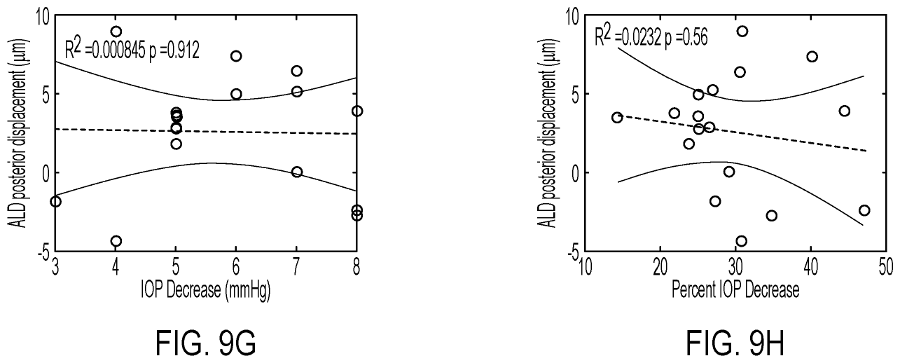

FIG. 9A, FIG. 9B, FIG. 9C, FIG. 9D, FIG. 9E, FIG. 9F, FIG. 9G, and FIG. 9H show plots of strain magnitudes increased with greater IOP decrease and greater percent IOP decrease in the medication change eyes of Group 1. Greater strain magnitudes were more associated with percent IOP decrease than with IOP decrease magnitude. FIG. 9A shows maximum principal strain, Eₘₐₓ, increased with greater IOP decrease. FIG. 9B shows maximum principal strain, Eₘₐₓ, increased with greater percent IOP decrease. FIG. 9C shows maximum shear strain, Γₘₐₓ, increased with greater IOP decrease. FIG. 9D shows maximum shear strain, Γₘₐₓ, increased with greater percent IOP decrease. FIG. 9E shows compressive Eᵣᵣ increased with greater IOP decrease. FIG. 9F shows compressive Eᵣᵣ increased with greater percent IOP decrease. FIG. 9G shows ALD did not change with IOP decrease magnitude. FIG. 9H shows ALD did not change with percent IOP decrease.

Further linear regression tests were conducted to investigate whether strains and ALD change magnitude were associated with percent IOP decrease. For the entire medication change group, greater Γₘₐₓ was associated with greater IOP percent decrease (R²=0.222, p=0.023) and greater Eₘₐₓ was borderline associated with greater IOP percent decrease (R²=0.125, p=0.099). A more negative (compressive) Eᵣᵣ correlated with greater IOP percent decrease (R²=0.234, p=0.019) while E_{zz}, Eₜₜ, Eᵣₜ, E_{tz}, E_{rz}, and ALD change were not significantly associated with IOP percent decrease. In Group 1, there was a stronger positive correlation between greater Eₘₐₓ with greater percent IOP decrease than there was for greater Eₘₐₓ with greater IOP decrease magnitude (FIG. 9A). The same relationship was observed even more strikingly for Γmax and percent IOP decrease compared to Γmax and IOP decrease magnitude (FIG. 9C). In Group 1, greater compressive Eᵣᵣ was also associated with greater IOP percent decrease. E_{zz}, Eₜₜ, Eᵣₜ, E_{tz}, E_{rz}, and ALD change magnitude were not associated with percent IOP decrease.

Linear regression analysis was conducted to investigate whether strain magnitude was related to baseline IOP. For the entire medication change group and for Group 1, there were no significant or borderline significant associations between E_{zz}, Eᵣᵣ, Eₜₜ, E_{rz}, E_{zt}, or Eᵣₜ with baseline IOP. Furthermore, neither Eₘₐₓ, Γₘₐₓ, nor ALD change was associated with baseline IOP.

Linear regression analysis was used to evaluate the relationship between ALC strains and ALD change with three clinically relevant measures of glaucoma damage: average RNFL, MD, and VFI. For the entire medication change group, greater Eₘₐₓ and greater Γmax were associated with a thicker average RNFL (R²=0.174, p=0.048 and R²=0.180, p=0.043 respectively). Eᵣᵣ, Eₜₜ, E_{rz}, Eᵣₜ, E_{tz}, and ALD change were not related to average RNFL. For Group 1, similar behavior was observed. Greater tensile E_{zz}, greater Eₘₐₓ, and greater Γₘₐₓ were associated with having a thicker average RNFL (R²=0.425, p=4.55E-3; R²=0.424, p=4.61E-3; and R²=0.286, p=0.027; respectively). For the entire medication change group, there was a borderline negative correlation between E_{zz} and MD, suggesting tensile E_{zz} increases with lower (worse) MD (R²=0.126, p=0.096). There were no significant correlations between Eₘₐₓ, Γₘₐₓ, Eᵣᵣ, Eₜₜ, E_{rz}, E_{tz}, Eᵣₜ, or ALD change with MD. For Group 1, there were no associations between Eₘₐₓ, Γₘₐₓ, E_{zz}, Eᵣᵣ, Eₜₜ, E_{rz}, E_{tz}, Eᵣₜ, or ALD change and MD. In both the entire medication change group and Group 1, there were no associations between any of the strains or ALD change with VFI.

Simple linear regression analysis revealed greater strain compliance responses for Eₘₐₓ, Γₘₐₓ, and E_{zz} with greater RNFL thickness for the eyes in Group 1 (R²=0.415, p=5.26 E-4; R²=0.262, p=0.036; and R²=0.376, p=5.26E-4; respectively). The strain compliance responses for Eᵣᵣ, Eₜₜ, E_{rz}, Eᵣₜ, E_{zt}, and ALD change were not correlated with average RNFL. For Group 1, the opposite trends were observed when linear regression analysis was used to evaluate the relationship between strain compliance and MD and the relationship between strain compliance and VFI. More compliant strain response for Eₘₐₓ, Γₘₐₓ, and E_{zz} was associated with lower MD. Likewise, a greater strain compliance response for Eₘₐₓ, and Γₘₐₓ was associated with lower VFI. These regression tests were only conducted for Group 1 because the strain compliance of Group 2 and the entire medication change group could not be computed due to the fact these groups contain eyes that did not undergo IOP change which would result in division by zero.

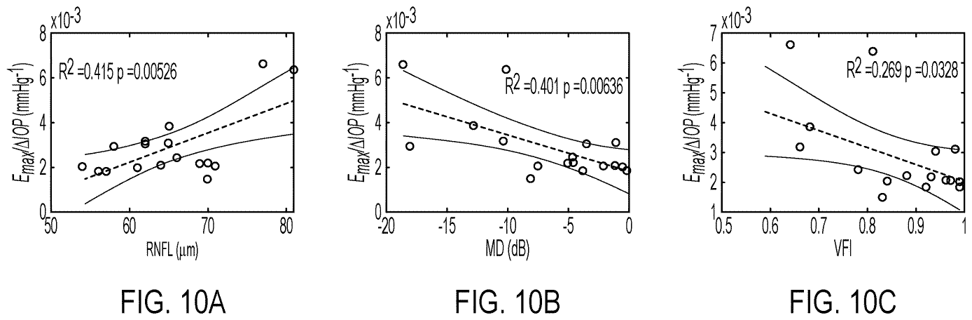

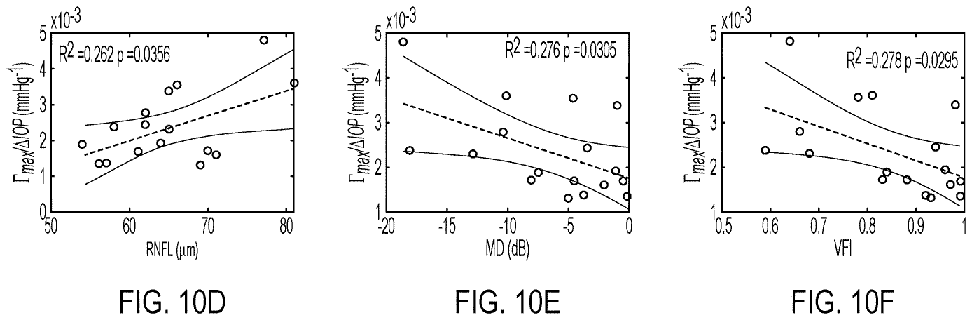

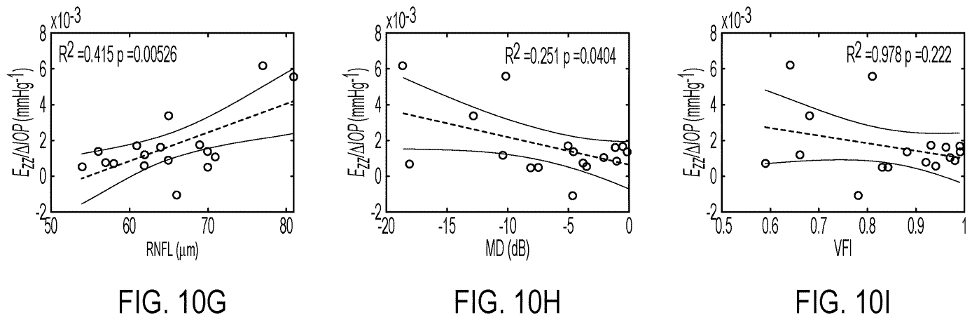

FIG. 10A, FIG. 10B, FIG. 10C, FIG. 10D, FIG. 10E, FIG. 10F, FIG. 10G, FIG. 10H, and FIG. 10I show plots of linear regression analysis was used to test whether strain compliance response to IOP change was associated with glaucoma damage. In Group 1, more compliant strain response for Eₘₐₓ, Γₘₐₓ, and E_{zz} was associated with a thicker average RNFL, however, a more compliant strain response for Eₘₐₓ, Γₘₐₓ, and VFI was associated with lower MD and lower VFI.

For the entire medication change group, there were no significant or borderline significant associations between Eₘₐₓ, Γₘₐₓ, E_{zz}, Eᵣᵣ, Eₜₜ, E_{rz}, E_{tz}, Eᵣₜ, or ALD change with age. For Group 1, there was a borderline association of greater compressive Eᵣᵣ with greater age (R²=0.229, p=0.052), however, this trend may be influenced by an outlier by age. This outlier had an age of 30 while the rest of the of the samples were from the eyes of patients whose age ranged from roughly 50 to 80 years old. For Group 1, the rest of the strains (Eₘₐₓ, Γₘₐₓ, E_{zz}, Eₜₜ, E_{rz}, E_{tz}, Eᵣₜ) and ALD change did not vary significantly with age.

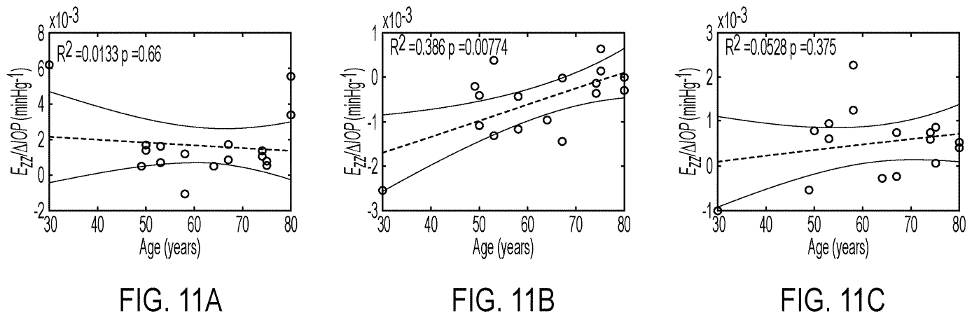

For Group 1, there was a more compliant compressive Eᵣᵣ strain response with younger age (R²=0.386, p=7.74E-3), however, there appeared to be an outlier with an age of 30 years old. If this outlier were removed, it appears from FIG. 11B that there would be little to no correlation between Eᵣᵣ strain compliance and age for the narrow age range. There were no associations between the strain compliance response for E_{zz}, Eₜₜ, E_{rz}, E_{tz}, Eᵣₜ, Eₘₐₓ, Γₘₐₓ, or ALD with age. Additionally, there was no association between IOP percent decrease and age.

FIG. 11A, FIG. 11B, and FIG. 11C show plots of linear regression tested whether strain compliance response was correlated with age in Group 1, where FIG. 11A shows strain compliance response for E_{zz}, FIG. 11B shows strain compliance response for Eᵣᵣ, FIG. 11C shows ALD change compliance response. Regression analysis suggested there was a greater compressive Eᵣᵣ strain compliance response with younger age, however this trend appears to be anchored by an age outlier with an age of 30 years old. The E_{zz} strain and ALD change compliance responses were not correlated with age.

Simple linear regression tests were used to assess the relationship between three measures of glaucoma damage and age. For the entire medication change group, neither average RNFL nor VFI were associated with age. MD was borderline associated with age such that higher MD (less damage) was associated with greater age. This trend, however, appeared to be caused by an age outlier with an age of 30 years old. For Group 1, average RNFL, MD, and VFI were not significantly related to age.

Simple linear regression was used to assess whether MD and VFI varied with average RNFL. Neither MD nor VFI was associated with average RNFL for the entire medication change group or for Group 1, suggesting these two measures of functional damage—VFI and MD—were not associated with the overall structural damage represented by average RNFL thickness.

According to the disclosed experiments, there were 6 eyes from 5 patients whose IOP only decreased by −0.3±0.8 mmHg. The minimal decrease in IOP for these Group 2 eyes suggests the medication type was either ineffective for the particular patient or, perhaps, suggests lack of patient compliance. For various reasons, including cost, forgetfulness, or patient reluctance (some topical eye drops may cause mild discomfort), the patient compliance rate for taking glaucoma eye drops is relatively low. The patient compliance rate tends to decrease with time, falling below 50% after one year. As this was a short-term study with a treatment period of approximately one week, patient compliance was relatively higher, perhaps due to the “white-coat adherence” effect, with only six eyes (26% of the sample size) from five patients suggesting poor compliance.

Linear regression analysis found that greater magnitude of Eₘₐₓ and Γₘₐₓ was more highly correlated with greater IOP percent decrease rather than IOP decrease magnitude, suggesting the ALC strain response to IOP change is not linear. This result was consistent with the findings in the study by Midgett et al. and was not surprising since IOP change alone is not normalized and it is the level of IOP rather than “elevated” IOP that is associated with greater glaucoma risk. Unlike the suturelysis eyes in previous studies by our group, there was no association between ALD change and greater strains for the medication change eyes nor was there an association between ALD change and IOP decrease or IOP percent decrease. Additionally, there were no associations between ALD change or strains and baseline IOP for the medication change eyes in the current study, whereas previous work by our group suggested ALD change is associated with having a lower baseline IOP. This could perhaps be due to the difference in the patient population. The eyes of patients undergoing post trabeculectomy suturelysis likely have greater glaucoma damage compared to the eyes of patients subjected to IOP lowering through glaucoma eye drops.

In both the entire medication change group and Group 1, IOP change related to medication change produced significant mean tensile E_{zz} strain that exceeded baseline error but not correlation error. Due to lack of usable control data, Group 2 was used to function as a “pseudo-control” group for IOP change. After negligible IOP change (0 to 1 mmHg), the eyes in Group 2 still had a significant tensile E_{zz} strain. However, the magnitude of E_{zz} for Group 2 (0.003±0.003, p=0.054) was about three times smaller than E_{zz} for Group 1 (0.010±0.011, p=0.003). Additionally, E_{zz} for Group 2 did not exceed baseline error, unlike Group 1, suggesting the small E_{zz} strains in Group 2 may be due to error such as patient head or eye movement between images taken back-to-back, rather than IOP change. Eᵣᵣ for Group 2 was not significantly different from zero or greater than baseline and correlation error, unlike Eᵣᵣ for Group 1. These results suggest the methods used in the present study are valid for measuring repeatable strains in the LC. However, future studies should repeat these experiments using proper controls (i.e. eyes of patients not undergoing medication change).

The current study showed that the ALC of glaucoma eyes that underwent an average IOP decrease of 6 mmHg (Group 1) due to starting hypotensive eyedrops had significant tensile E_{zz} strains and compressive Eᵣᵣ strains. This was the expected behavior for E_{zz}, since decreasing IOP reduces the load carried by the LC along the Z-direction, allowing the tissue to decompress and expand axially. A compressive Eᵣᵣ implies the LC contracted radially. These findings are consistent with previous studies by our group which found that IOP reduction via post trabeculectomy suturelysis produces tensile E_{zz} and compressive Eᵣᵣ strains in the ALC. Not only were the directions of E_{zz} and Eᵣᵣ consistent for the medication change eyes and the suturelysis eyes, but the average magnitudes of these strains were comparable, despite the medication change eyes undergoing an average IOP decrease roughly half that of the suturelysis eyes.

The medication-change eyes in this study underwent a small yet significant ALD change that was positive on average. This was not only true for Group 1 but for all study eyes as well, which had an even smaller average IOP decrease of approximately 4 mmHg. This suggests that even relatively small IOP reductions can cause ALD change over the course of one week. The ALC surface may move in either direction along the z-axis, however, on average it moves posteriorly, moving deeper inside the eye. The studies by Quigley et al. and Czerpak et al. found that the LC surface can move in either direction 20 minutes after IOP-lowering through suturelysis but found no significant ALD change. The study by Kim et al. showed ALD depth decreases from baseline by 9.13±2.05 μm after IOP-lowering through eye drops over the course of one year, in other words, they found that the ALC surface moves anteriorly, towards the front of the eye. In contrast, the current study found a small but significant positive ALD change, indicating LC surface movement that is posterior on average for medication change eyes after one week. The difference in these results may be due to remodeling of the LC over time.

One difference between the methods used in this study and those used in others was the average time duration between acquiring pre- and post-treatment OCT images. The current study measured strains from IOP reduction over the course of one week as opposed to the studies by Czerpak et al. and Midgett et al. which measured strains from IOP decrease from suturelysis or IOP increase from tight-fitting goggles after 20 minutes. From a biomechanical standpoint, this suggests that another independent variable may be at play time.

The longer time duration between each imaging session for the medication-change eyes could allow for slowly increasing strains assuming a relatively constant load (stable IOP after lowering), implying a creep response characteristic of viscoelastic materials. Biological tissues are viscoelastic in nature, meaning their deformation response behaves partly like a viscous fluid and partly like an elastic solid. Creep is the slow increase in strain with time when the loading conditions are held constant. The ALC E_{zz} strain (0.010±0.011, p=0.003) and Eᵣᵣ strain (0.003±0.005, p=0.012) magnitudes for the Group 1 eyes were comparable to the ALC E_{zz} (0.0094±0.0012, p=0.012) and Eᵣᵣ (−0.0019±0.0033, p=0.0043) for the suturelysis study by Czerpak et al., despite having roughly half the average IOP decrease of the suturelysis eyes. This could be explained by the event of a creep response occurring in the Group 1 eyes, which were subjected to an assumed constant load (stable IOP after lowering) over a one-week period as opposed to a 20-minute period, in which, likely only the elastic strain response occurred. A creep response to IOP lowering suggests that the LC is a viscoelastic material.

One result from the current study was that greater strains and more compliant strain response were associated with thicker RNFL for the medication-change eyes. This was the opposite relationship observed for the suturelysis eyes, in which more compliant strain response was associated with thinner RNFL (more damage). However, consistent with the findings from the suturelysis strain response study, greater strains and more compliant strain response in the medication-change eyes were associated with lower VFI and more negative MD. One possible explanation for the conflicting relationships observed in the medication-change eyes is that VFI and MD are age-normalized measures of glaucoma damage, whereas average RNFL is not normalized for age and may not be a suitable measure of damage for this analysis. However, although average RNFL is expected to decrease with age, linear regression showed average RNFL did not vary by age for the medication change eyes. Average RNFL is also expected to decrease with increasing damage, but linear regression revealed RNFL in the present study was also not associated with worse MD or VFI.

The medication change eyes in the study by Kim et al. found greater reduction of LC curvature was associated with younger age. The present study did not measure LC curvature but found ALD change was not significantly correlated with age. Eᵣᵣ strain was more compliant with lower age, however it was unclear whether this trend was truly significant since it appeared to be anchored by an age outlier with a much younger age and more compliant Eᵣᵣ response (FIG. 11B).

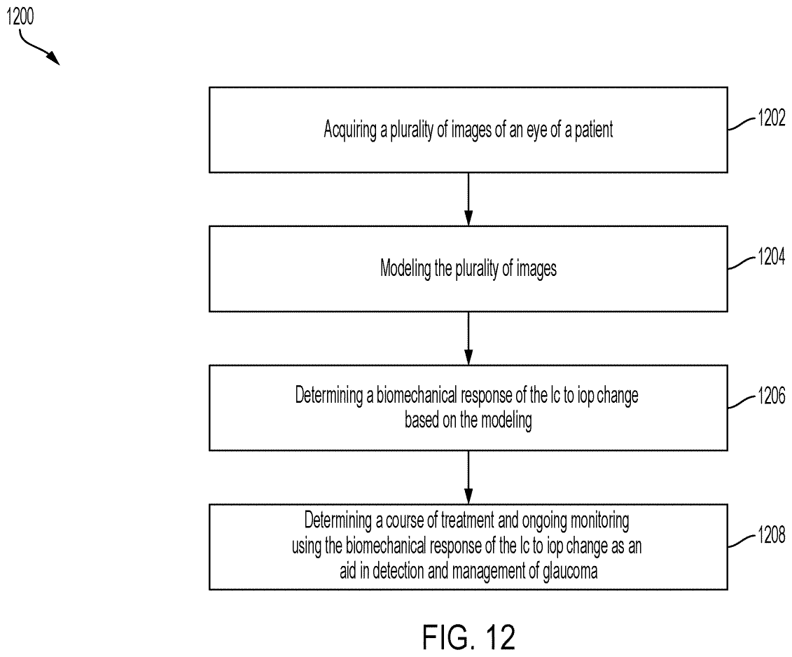

FIG. 12 shows a flowchart of a method 1200 to characterize in vivo biomechanical response of a lamina cribrosa (LC) to intraocular pressure (IOP) change as a result of a change in glaucoma medication. The method 1200 comprises acquiring a plurality of images of an eye of a patient, as in 1202. For example, the plurality of images comprise optical coherence tomography (OCT) scans.

The method 1200 continues by modeling the plurality of images, as in 1204. For example, the modeling comprises stacking consecutive OCT scans to for a 3D image volume. For example, the modeling comprises performing a digital volume correlation (DVC) operation. For example, the modeling comprises performing a digital volume correlation (DVC) operation. For example, the modeling comprises modeling a relationship of LC strains and strain compliance to measures of glaucoma damage, wherein the measures of glaucoma damage comprise RNFL thickness, mean deviation (MD), and visual field index (VFI).

For example, the OCT scans comprise a brightness scan (B-scan), wherein the B-scan comprises data from scanning a sample laterally at multiple points that results in a grayscale cross-sectional image where a grayscale value at each point represents a brightness amplitude of light reflected from a portion of the eye. For example, the OCT scans comprise acquired B-scans spaced at equal intervals circumferentially around an optic nerve head that results in 2D cross-sectional images in which a height of a scan represented an axial direction and a length of the scan represented a radial direction. For example, the OCT scans comprise spectral domain OCT (SDOCT).

The method 1200 continues by determining a biomechanical response of the LC to IOP change based on the modeling, as in 1206. For example, the biomechanical response is short-term biomechanical strain response. For example, the short-term biomechanical strain response is about one week.

The method 1200 continues by determining an intensity of a course of treatment and ongoing monitoring using the biomechanical response of the LC to IOP change as an aid in detection and management of glaucoma, as in 1208. For example, the course of treatment comprises a glaucoma medication such as the degree to which IOP would be lowered by IOP-lowering eye drops.

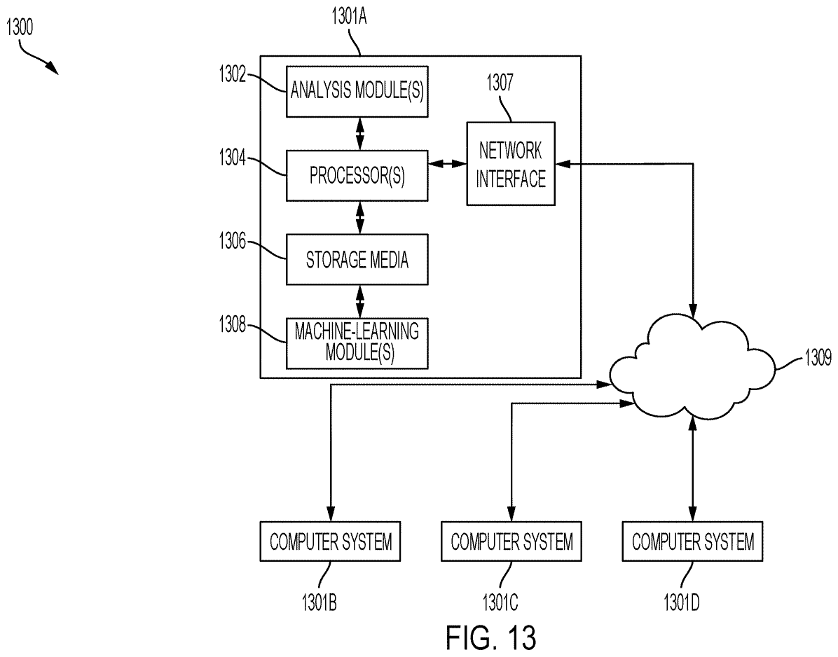

In some embodiments, any of the methods of the present disclosure may be executed by a computing system. FIG. 13 illustrates an example of such a computing system 900, in accordance with some embodiments. The computing system 1300 may include a computer or computer system 1301A, which may be an individual computer system 1301A or an arrangement of distributed computer systems. The computer system 1301A includes one or more analysis module(s) 1302 configured to perform various tasks according to some embodiments, such as one or more methods disclosed herein. To perform these various tasks, the analysis module 1302 executes independently, or in coordination with, one or more processors 1304, which is (or are) connected to one or more storage media 906. The processor(s) 1304 is (or are) also connected to a network interface 1307 to allow the computer system 1301A to communicate over a data network 1309 with one or more additional computer systems and/or computing systems, such as 1301B, 1301C, and/or 1301D (note that computer systems 1301B, 1301C and/or 1301D may or may not share the same architecture as computer system 1301A, and may be located in different physical locations, e.g., computer systems 1301A and 1301B may be located in a processing facility, while in communication with one or more computer systems such as 1301C and/or 1301D that are located in one or more data centers, and/or located in varying countries on different continents).

A processor can include a microprocessor, microcontroller, processor module or subsystem, programmable integrated circuit, programmable gate array, or another control or computing device.

The storage media 1306 can be implemented as one or more computer-readable or machine-readable storage media. The storage media 1306 can be connected to or coupled with a machine learning module(s) 1308. Note that while in the example embodiment of FIG. 13 storage media 1306 is depicted as within computer system 1301A, in some embodiments, storage media 1306 may be distributed within and/or across multiple internal and/or external enclosures of computing system 1301A and/or additional computing systems. Storage media 1306 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 1300 is only one example of a computing system, and that computing system 1300 may have more or fewer components than shown, may combine additional components not depicted in the example embodiment of FIG. 13, and/or computing system 1300 may have a different configuration or arrangement of the components depicted in FIG. 13. The various components shown in FIG. 9 may be implemented in hardware, software, or a combination of both hardware and software, including one or more signal processing and/or application specific integrated circuits.

Further, the steps in the processing methods described herein may be implemented by running one or more functional modules in an information processing apparatus such as general purpose processors or application specific chips, such as ASICs, FPGAs, PLDs, or other appropriate devices. These modules, combinations of these modules, and/or their combination with general hardware are all included within the scope of protection of the invention.

Models and/or other interpretation aids may be refined in an iterative fashion; this concept is applicable to embodiments of the present methods discussed herein. This can include use of feedback loops executed on an algorithmic basis, such as at a computing device (e.g., computing system 1300, FIG. 13), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

1. A method to characterize in vivo biomechanical response of a lamina cribrosa (LC) to intraocular pressure (IOP) change as a result of a change in glaucoma medication, the method comprising:
acquiring a plurality of images of an eye of a patient;
modeling the plurality of images;
determining a biomechanical response of the LC to IOP change based on the modeling; and
determining an intensity of a course of treatment and ongoing monitoring using the biomechanical response of the LC to IOP change as an aid in detection and management of glaucoma.

2. The method of claim 1, wherein the biomechanical response is short-term biomechanical strain response.

3. The method of claim 2, wherein the short-term biomechanical strain response is about one week.

4. The method of claim 1, wherein the treatment comprises glaucoma medication, wherein the glaucoma medication comprises IOP-lowering eye drops.

5. The method of claim 1, wherein the plurality of images comprises optical coherence tomography (OCT) scans.

6. The method of claim 5, wherein the modeling comprises stacking consecutive OCT scans to for a 3D image volume.

7. The method of claim 5, wherein the OCT scans comprise a brightness scan (B-scan), wherein the B-scan comprises data from scanning a sample laterally at multiple points that results in a grayscale cross-sectional image where a grayscale value at each point represents a brightness amplitude of light reflected from a portion of the eye.

8. The method of claim 7, wherein the OCT scans comprise acquired B-scans spaced at equal intervals circumferentially around an optic nerve head that results in 2D cross-sectional images in which a height of a scan represented an axial direction and a length of the scan represented a radial direction.

9. The method of claim 5, wherein the OCT scans comprise spectral domain OCT (SDOCT).

10. The method of claim 1, wherein the modeling comprises performing a digital volume correlation (DVC) operation.

11. The method of claim 1, wherein the modeling comprises modeling a relationship of LC strains and strain compliance to measures of glaucoma damage, wherein the measures of glaucoma damage comprise RNFL thickness, mean deviation (MD), and visual field index (VFI).

12. A system to characterize in vivo biomechanical response of a lamina cribrosa (LC) to intraocular pressure (IOP) change as a result of a change in glaucoma medication, the system comprising:
an imaging system to acquire a plurality of images of an eye of a patient;
a computer system that is configured to perform operations comprising:
modeling the plurality of images;
determining a biomechanical response of the LC to IOP change based on the modeling; and
determining an intensity of a course of treatment and ongoing monitoring using the biomechanical response of the LC to IOP change as an aid in detection and management of glaucoma.

13. The system of claim 12, wherein the biomechanical response is short-term biomechanical strain response.

14. The system of claim 13, wherein the short-term biomechanical strain response is about one week.

15. The system of claim 12, wherein the treatment comprises glaucoma medication, wherein the glaucoma medication comprises IOP-lowering eye drops.

16. The system of claim 12, wherein the plurality of images comprises optical coherence tomography (OCT) scans.

17. The system of claim 16, wherein the modeling comprises stacking consecutive OCT scans to for a 3D image volume.

18. The system of claim 16, wherein the OCT scans comprise a brightness scan (B-scan), wherein the B-scan comprises data from scanning a sample laterally at multiple points that results in a grayscale cross-sectional image where a grayscale value at each point represents a brightness amplitude of light reflected from a portion of the eye.

19. The system of claim 16, wherein the OCT scans comprise acquired B-scans spaced at equal intervals circumferentially around an optic nerve head that results in 2D cross-sectional images in which a height of a scan represented an axial direction and a length of the scan represented a radial direction.

20. The system of claim 16, wherein the OCT scans comprise spectral domain OCT (SDOCT).

21. (canceled)

22. (canceled)
